(19) United States
(12) Patent Application Publication
(10) Pub. No.: US 2018/0036433 A1
(43) Pub. Date: Feb. 8, 2018

(54) COMPOUNDS FOR THE DIAGNOSIS OR TREATMENT OF DISORDERS ASSOCIATED WITH PROTEIN OR PEPTIDE OLIGOMERS

(71) Applicants: Agency for Science, Technology and Research, Singapore (SG); National University of Singapore, Singapore (SG); New York University, New York, NY (US)

(72) Inventors: Young-Tae Chang, Singapore (SG); Dongdong Su, Singapore (SG); Chai Lean Teoh, Singapore (SG); Srikanta Sahu, Singapore (SG); Thomas Wisniewski, Staten Island, NY (US)

(21) Appl. No.: 15/523,999

(22) PCT Filed: Nov. 3, 2015
(86) PCT No.: PCT/US2015/058692

§ 371 Date: May 3, 2017

(30) Foreign Application Priority Data
Nov. 3, 2014 (SG) 10201407182R

(51) Int. Cl. A61K 49/00; G01N 33/68; C07F 5/02

(52) U.S. Cl. CPC A61K 49/0021; C07F 5/022; G01N 33/6896; G01N 2333/4709

(57) ABSTRACT

The present invention relates to compounds of Formula (I) as defined herein as well as conjugates comprising said compounds and pharmaceutical compositions comprising said compounds or conjugates. Also encompassed are methods for selectively detecting oligomeric proteins or peptides associated with a conformational disorder in a sample, and methods for diagnosing or treating a conformational disorder associated with protein or peptide oligomers in a subject.

### CROSS-REFERENCE TO RELATED APPLICATION

This application makes reference to and claims the benefit of priority of a Singapore Provisional Application for “A Chemical Fluorescent Probe for the Detection of Amyloid 13-Peptide Oligomers” filed on Nov. 3, 2014, and duly assigned application number 10201407182R. The content of said application filed on Nov. 3, 2014, is incorporated herein by reference for all purposes, including an incorporation of any element or part of the description, claims or drawings not contained herein and referred to in Rule 20.5(a) of the PCT, pursuant to Rule 4.18 of the PCT.

### FIELD OF THE INVENTION

The present invention relates to compounds selective for oligomeric proteins or peptides associated with conformational disorders, conjugates and pharmaceutical compositions containing them as well as methods for diagnosing or treating the conformational disorders.

### BACKGROUND OF THE INVENTION

Neurodegenerative disorders are associated with conditions in which neuronal cells deteriorate, lose function, and often die. As they are generally progressive, the consequences of neurodegenerative disorders are often devastating. Patients with neurodegenerative disorders may suffer severe deterioration in cognitive or motor skills. As a result, their quality of life and life expectancy may be considerably reduced. In humans, these diseases include, but are not limited to, Alzheimer's Disease (AD) and prion diseases. Most neurodegenerative disorders are classified as “conformational” disorders in that their pathogenesis is related to a structural change of a normal self protein or peptide into an oligomeric form with a high β-sheet content that is associated with neurotoxicity.

Along with aging of the world's population and the growing epidemic of neurodegenerative disorders, an early detection of these disorders becomes ever more critical for evaluating risk, assessing new therapies, and treating them with early intervention. Unfortunately, both diagnostic and therapeutic options for neurodegenerative disorders still remain largely limited.

AD is one of the most studied neurodegenerative disorders in which amyloid β-peptides (Aβ) aggregate forming extracellular neuritic plaques in the brain. AD affects well over 35 million worldwide and this number is expected to grow dramatically as the population ages (Brookmeyer, R., et al. Alzheimers Dement, 2011, 7, 61). Amyloidogenic proteins and peptides can adopt a number of distinct assembly states and a key issue is which of these assembly states is more closely associated with pathogenesis. Fibrillization of Aβ resulting in plaque deposition has long been regarded as the cause of neurodegeneration in AD. However, recent data suggest that oligomeric soluble Aβ is principally responsible for the pathogenesis of AD and its levels are more important in the disease progression (Haass, C. and Selkoe, D. J. Nat. Rev. Mol. Cell. Biol. 2007, 8, 101; Caughey, B. and Lansbury, P. T. Annu. Rev. Neurosci. 2003, 26, 267; Walsh, D. M. and Selkoe, D. J. J. Neurochem. 2007, 101, 1172; Rijal Upadhaya, A., et al. Brain, 2014, 137, 887). The concept of the involvement of Aβ intermediates in the development of AD has been used to explain why amyloid pathology, defined by Aβ plaque load, is only poorly correlated with clinical AD presentation, effectively suggesting that amyloid plaque is a relatively non-toxic aggregated form of Aβ. Hence, there is an urgent need for the development of detection methods that are able to identify a variety of morphologically distinct Aβ peptides.

Aβ plaques have been detected using a number of fibril-specific dyes, such as Congo Red (CR) or Thioflavin T (ThT) (Westermark, G. T., et al. Methods Enzymol. 1999, 309, 3), which preferably bind to mature amyloid fibrils. Neither CR nor ThT are suitable for in vivo use, nonetheless they serve as the basis for the development of improved imaging agents for detecting amyloid accumulation and have led to the development of compounds such as PiB (Klunk, W. E., et al. Ann. Neurol. 2004, 55, 306). Despite extensive research for many decades, it was only until recently that a brain imaging agent, Florbetapir was approved by the Food and Drugs Administration (FDA) to evaluate AD (Yang, L., et al. N. Engl. J. Med., 2012, 367, 885). In recent years, however, there has been a paradigm shift with numerous reported efforts involved in the development of effective methods for detecting Aβ oligomers, including oligomer-specific antibody (Morgado, I., et al. Proc. Natl. Acad. Sci. USA 2012, 109, 12503), oligomer-specific peptide-FlAsh system (Hu, Y., et al. Chembiochem 2010, 11, 2409; Hu, Y., et al. Mol. Biosyst. 2012, 8, 2741), peptide-based fluorescent protein (Takahashi, T. and Mihara, H. Chem. Commun. 2012, 48, 1568) as well as ELISA (Bruggink, K. A., et al. Anal. Biochem. 2013, 433, 112). Yet, these detection methods often involve laborious work, complicated instrumentation or long testing time, which make them inconvenient to use. In addition, their inability to cross the blood-brain barrier (BBB) makes them inappropriate for in vivo applications. Small fluorescent probes with high sensitivity and easy visibility would offer a convenient and straightforward approach for the detection of Aβ oligomers. One of the reported oligomer-specific fluorescent probes can distinguish soluble Aβ from Aβ of ordered conformation but cannot discriminate oligomers from fibrils, and lacks demonstration of biological application capabilities (Jameson, L. P. and Dzyuba, S. V. Bioorg. Med. Chem. Lett. 2013, 23, 1732; Smith, N. W., et al. Biochem. Biophys. Res. Commun. 2010, 391, 1455).

Therefore, there remains a considerable need in the art for oligomer-specific diagnostic and/or therapeutic agents that overcome the drawbacks of existing techniques.

### SUMMARY OF THE INVENTION

The present invention satisfies the aforementioned need in the art by providing novel diagnostic and therapeutic agents selective for oligomeric proteins or peptides.

In a first aspect, the present invention provides a compound of Formula (I)

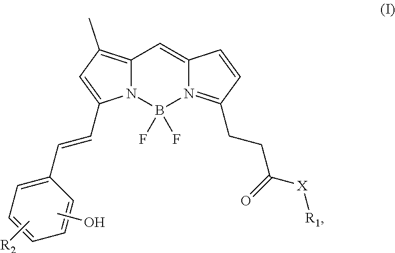

wherein:
X is a direct bond or is selected from the group consisting of —NR, —O—, and —S—; R is selected from the group consisting of H and a substituted or unsubstituted alkyl; and R₁ and R₂ are each independently selected from the group consisting of H, halogen, a substituted or unsubstituted alkyl, a substituted or unsubstituted alkoxy, a substituted or unsubstituted alkenyl, a substituted or unsubstituted alkynyl, a substituted or unsubstituted heterocycle, a substituted or unsubstituted aryl, a substituted or unsubstituted alkyl-aryl, a substituted or unsubstituted alkyl-heterocycle, a substituted or unsubstituted heteroaryl.

In some embodiments, X is —NH— or —O—.

In some embodiments, R₁ is a substituted or unsubstituted C₁-C₁₀ alkyl.

In some embodiments, R₁ is ethyl or propyl.

In some embodiments, R₁ is —CH₂CH₂F.

In some embodiments, R₁ is —CH₂CCl₃.

In some embodiments, R₁ is a substituted or unsubstituted C₇-C₁₀ alkyl-aryl.

In some embodiments, R₁ is benzyl.

In some embodiments, R₂ is at the 3-position (meta) and OH is at the 2-position (ortho) of the phenyl ring.

In some embodiments, R₂ is a substituted or unsubstituted C₁-C₁₀ alkoxy.

In some embodiments, R₂ is ethoxy.

In preferred embodiments, the compound of Formula (I) is selected from the group consisting of:

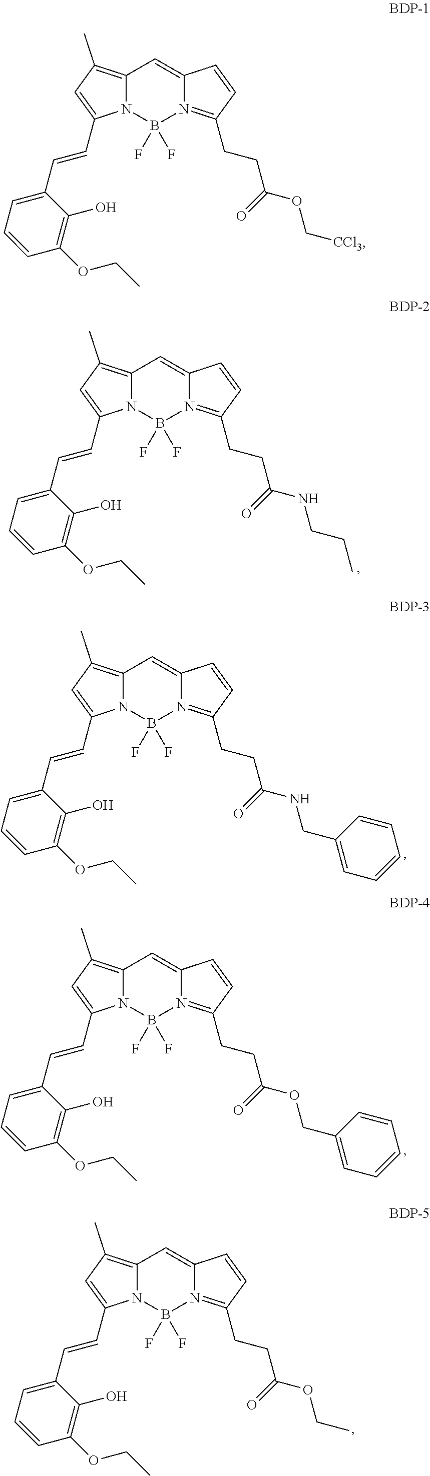

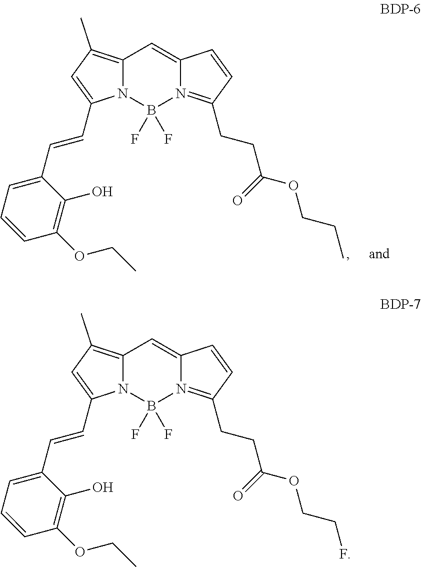

In a second aspect, the invention provides a conjugate comprising the compound of the invention and a detectable marker.

In a third aspect, the invention provides a pharmaceutical composition comprising the compound or conjugate of the invention, and a pharmaceutically acceptable carrier.

In a fourth aspect, the invention provides a method for selectively detecting oligomeric proteins or peptides associated with conformational disorders in a sample, the method comprising:

(a) contacting the sample with the compound or conjugate of the invention, wherein the compound binds selectively to the oligomers, under conditions that allow binding of the compound to the oligomers; and
(b) detecting the protein oligomers by measuring the signal of the complexes formed between the oligomers and said compound or conjugate.

In some embodiments, the oligomeric proteins or peptides are Aβ oligomers or PrP^{Sc} oligomers.

In a fifth aspect, the invention provides a method for diagnosing conformational disorders associated with protein or peptide oligomers in a subject, comprising administering to said subject an effective amount of the compound or conjugate of the invention and measuring the signal of the complexes formed between said oligomers and said compound or conjugate.

In a final aspect, the invention provides a method for treating or preventing a conformational disorder associated with protein or peptide oligomers in a subject, comprising administering to said subject an effective amount of the compound or pharmaceutical composition of the invention.

In various embodiments, the disorder is a neurodegenerative disorder.

In various embodiments, the neurodegenerative disease is Alzheimer's Disease (AD) or a prion disease.

In various embodiments, the subject is a mammal, preferably a human.

### BRIEF DESCRIPTION OF THE DRAWINGS

The invention will be better understood with reference to the detailed description when considered in conjunction with the non-limiting examples and the accompanying drawings.

FIG. 1 shows the conformational specificity of BDP-1. (a) Chemical structure of BDP-1. (b) Emission spectra of BDP-1 alone and when incubated with monomers, oligomers and fibrils of Aβ (λₑₓ=530 nm, dye: 5 μM, Aβ: 20 μM).

FIG. 2 shows the characterization of monomers, oligomers and fibrils formed from synthetic Aβ1-40 peptide. (a) Dot blots of Aβ probed by oligomer-specific A11 and 6E10 antibodies. (b) Emission spectra of ThT alone and when incubated with monomers, oligomers and fibrils of Aβ (λₑₓ=440 nm, dye: 5 μM, Aβ: 20 μM).

FIG. 3 shows the spectra and spectral information of BDP-1. (a) Absorbance and emission spectra of BDP-1. (b) Absorbance maximum, emission maximum and quantum yield of BDP-1, measured in DMSO.

FIG. 4 shows the BDP-1 binding constant (Aβ oligomers: 20 μM, λex=530 nm). F is the fluorescence intensity of BDP-1 at 580 nm after binding with Aβ oligomers; F0 is the fluorescence intensity of BDP-1 at 580 nm before binding with Aβ oligomers.

FIG. 5 shows the biophysical characterization of oligomer-specific response. (a) Time-dependent fibril formation of Aβ was monitored by ThT, whereas BDP-1 detects on-fibril pathway oligomers (dye: 5 μM, Aβ: 20 μM). (b) Kinetics of oligomer-specific immunoreactivity during fibrillogenesis, as probed by oligomer-specific A11 antibody and 6E10 antibody against Aβ (c) Pelleting assay for Aβ at various time-points after fibril formation time course have been initiated. (d) Transmission electron microscopy (TEM) images of Aβ at day-0, day-1 and day-4 of fibrillogenesis.

FIG. 6 shows the time-dependent fibril formation of Aβ as monitored by ThT and the on-fibril pathway oligomers as detected by BDP-1 (dye: 5 μM, Aβ: 20 μM). F is the fluorescence intensity of BDP-1 at 580 nm after binding with Aβ oligomers; F0 is the fluorescence intensity of BDP-1 at 580 nm before binding with Aβ oligomers.

FIG. 7 shows the biophysical characterization of oligomer-specific response as shown by CD spectra for Aβ at various time-points after the fibril formation is initiated.

FIG. 8 shows the complex of BDP-1 and Aβ oligomers. (a) Aβ oligomer from X-ray (4NTR) from Ref 29. (b) The optimized BDP-1 structure at the B3LYP/6-31G* level. (c) The simulated complex structure of BDP-1 and Aβ oligomer.

FIG. 9 shows the analysis of site-directed thermodynamics of the complex of BDP-1 and Aβ oligomer (Aβ₁₇₋₃₆). Residue-specific free energy values (Δf) are plotted for the free energy of Aβ oligomer with BDP-1 binding (fcomplex) relative to that of Aβ oligomer without BDP-1 (fAβ oligomer) for each residue.

FIG. 10 shows the structural-activity relationship study of BDP-1. (a) Chemical modifications of BDP-1 derivatives; (b) Emission ratio of BDP-1 and derivatives in the presence of Aβ oligomers, compared to when in the presence of monomers or fibrils; (c) The ability of BDP-1 and derivatives to detect oligomers during Aβ fibrillogenesis over time.

FIG. 11 shows that BDP-1 and BDP-7 labels Aβ oligomers in AD brain. Upper panel: Pre-fibrillar, oligomers of Aβ visualized with the A11 antibody (red) and ThS against amyloid plaques (green). Arrow denotes one example of plaque core, without A11 reactivity, whereas A11 staining around the peripheral is highlighted by dashed circle. Lower panels: BDP-1 and BDP-7 (yellow), two probes of the invention, show extensive overlap with A11 reactivity (red). Co-localization appears as yellow-orange color. Scale bar, 100 μm.

FIG. 12 shows the toxicity of BDP-1 treatment in N2a mouse neuroblastoma cells. Cells were treated with BDP-1 at different concentrations for 72 hours before being subjected to CellTiter 96 AQueoue Non-Radioactive Cell Proliferation Assay. (*p<0.001 versus control).

FIG. 13 shows the ex vivo binding of BDP-1 in 18 month-old AD mouse brains. a, b and c show fluorescence in the APP/PS1 mouse brain injected with BDP-1 using the channel for 6E10/4G8 antibody labeling, BDP-1 labeling and the merged image, respectively. Arrows indicate plaques with co-localization. Scale bar: 100 μm.

FIG. 14 shows the effects of BDP-1 treatment on the cognitive ability of Tg mice as measured by the radial arm maze test (*p<0.001, by Bonferroni's multiple comparisons test).

FIG. 15 shows the presence of BDP-1 in mouse brain after one-month treatment of BDP-1. The brain sections were also labeled immunohistochemically with antibodies to IBA1, a marker of microglia.

FIG. 16 shows the effects of BDP-1 treatment on PrP^{Sc} infection of N2a cells. (a) Western Blot of PrP^{Sc} in N2a cells treated with vehicle or BDP-1 at different concentrations. (b) Statistics of 3 independent experiments comparing the densities of the bands of vehicle treated N2a/22L cells (lane 2 in FIG. 16a) to N2a/22L cells treated with 10 μM BDP-1 (p<0.001, by two-tailed student's t-test).

### DETAILED DESCRIPTION OF THE INVENTION

The following detailed description refers to, by way of illustration, specific details and embodiments in which the invention may be practiced. These embodiments are described in sufficient detail to enable those skilled in the art to practice the invention. Other embodiments may be utilized and structural, and logical changes may be made without departing from the scope of the invention. The various embodiments are not necessarily mutually exclusive, as some embodiments can be combined with one or more other embodiments to form new embodiments.

Unless otherwise defined, all technical and scientific terms used herein have the same meaning as commonly understood by one of ordinary skill in the art. In case of conflict, the present document, including definitions, will control.

The object of the present invention is to provide compounds selective for oligomeric proteins or peptides associated with a conformational disorder for the diagnosis or treatment thereof.

To this end, in a first aspect, the present invention provides a compound of Formula (I)

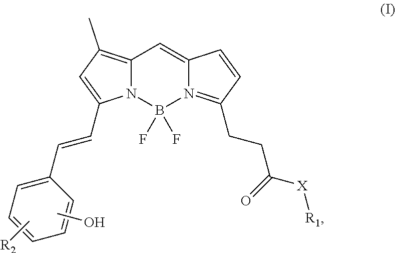

wherein:
X is a direct bond or is selected from the group consisting of —NR, —O—, and —S—; R is selected from the group consisting of H and a substituted or unsubstituted alkyl; and R₁ and R₂ are each independently selected from the group consisting of H, halogen, a substituted or unsubstituted alkyl, a substituted or unsubstituted alkoxy, a substituted or unsubstituted alkenyl, a substituted or unsubstituted alkynyl, a substituted or unsubstituted heterocycle, a substituted or unsubstituted aryl, a substituted or unsubstituted alkyl-aryl, a substituted or unsubstituted alkyl-heterocycle, a substituted or unsubstituted heteroaryl.

The term “alkyl” refers to a linear, branched, or cyclic saturated hydrocarbon group.

The term “halogen” refers to fluoro, chloro, bromo, and iodo.

The term “alkoxy” intends an alkyl group bound through a single, terminal ether linkage; that is, an “alkoxy” group may be represented as —O-alkyl where alkyl is as defined above.

The term “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although not necessarily, alkenyl groups herein contain 2 to about 18 carbon atoms, preferably 2 to 12 carbon atoms.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein contain 2 to about 18 carbon atoms, preferably 2 to 12 carbon atoms.

The term “heterocycle”, as used herein as a substituent is defined as including an aromatic or non aromatic cyclic alkyl, alkenyl, aryl or alkynyl moiety as defined above, having at least one O, S, P and/or N atom interrupting the carbocyclic ring structure. The term “heterocycle” also includes bicyclic, tricyclic and tetracyclic, spino groups in which any of the above heterocyclic rings is fused to one or two rings independently selected from an aryl ring, a cyclohexane ring, a cyclohexene ring, a cyclopentane ring, a cyclopentene ring or another monocyclic heterocyclic ring or where a monocyclic heterocyclic group is bridged by an alkylene group.

The term “alkyl-aryl” refers to an alkyl moiety bound to an aryl moiety.

The term “alkyl-heterocycle” refers to an alkyl moiety bound to a heterocycle moiety.

The term “heteroaryl” refers to an aryl moiety wherein at least one of its carbon atoms has been replaced with a heteroatom (e.g., N, O or S).

By “substituted” as in “substituted alkyl,” “substituted aryl,” and the like, as alluded to in some of the aforementioned definitions, is meant that in the alkyl, aryl, or other moiety, at least one hydrogen atom bound to a carbon (or other) atom is replaced with one or more non-hydrogen substituents. Examples of such substituents include, without limitation: functional groups such as halo, hydroxyl, sulfhydryl, C₁-C₂₄ alkoxy, C₂-C₂₄alkenyloxy, C₂-C₂₄alkynyloxy, C₅-C₂₀ aryloxy, acyl (including C₂-C₂₄ alkylcarbonyl (—CO-alkyl) and C₆-C₂₀arylcarbonyl (—CO-aryl)), acyloxy (—O-acyl), C₂-C₂₄ alkoxycarbonyl (—(CO)—O-alkyl), C₆-C₂₀aryloxycarbonyl (—(CO)—O-aryl), halocarbonyl (—CO)—X where X is halo), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), mono-(C₁-C₂₄alkyl)-substituted carbamoyl (—(CO)—NH(C₁-C₂₄ alkyl)), di-(C₁-C₂₄alkyl)-substituted carbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), cyano (—C≡N), isocyano (—N⁺≡C⁻), cyanato (—O—C≡N), isocyanato (—O—N⁺≡C⁻), isothiocyanato (—S—C≡N), azido (—N═N⁺═N⁻), formyl (—(CO)—H), thioformyl (—(CS)—H), amino (—NH₂), mono- and di-(C₁-C₂₄alkyl)-substituted amino, mono- and di-(C₅-C₂₀aryl)-substituted amino, C₂-C₂₄alkylamido (—NH—(CO)-alkyl), C₆-C₂₀arylamido (—NH—(CO)-aryl), imino (—CR═NH where R=hydrogen, C₁-C₂₄alkyl, C₅-C₂₀aryl, C₆-C₂₄alkaryl, C₆-C₂₄ aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂OH), sulfonate (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀arylsulfinyl (—(SO)-aryl), C₁-C₂₄ alkylsulfonyl (—SO₂-alkyl), C₅-C₂₀arylsulfonyl (—SO₂-aryl), phosphono (—P(O)(OH)₂), phosphonato (—P(O)(O⁻)₂), phosphinato (—P(O)(O⁻)), phospho (—PO₂), and phosphino (—PH₂); and the hydrocarbyl moieties C₁-C₂₄ alkyl (preferably C₁-C₁₈ alkyl, more preferably C₁-C₁₂ alkyl, most preferably C₁-C₆ alkyl), C₂-C₂₄ alkenyl (preferably C₂-C₁₈ alkenyl, more preferably C₂-C₁₂ alkenyl, most preferably C₂-C₆ alkenyl), C₂-C₂₄ alkynyl (preferably C₂-C₁₈ alkynyl, more preferably C₂-C₁₂ alkynyl, most preferably C₂-C₆ alkynyl), C₅-C₂₀ aryl (preferably C₅-C₁₄ aryl), C₆-C₂₄ alkaryl (preferably C₆-C₁₈ alkaryl), and C₆-C₂₄ aralkyl (preferably C₆-C₁₈ aralkyl). In addition, the aforementioned functional groups may, if a particular group permits, be further substituted with one or more additional functional groups. By “(Cₓ-C_{y})” (x and y being two different integers) is meant that the group contains x to y carbon atoms.

The term “unsubstituted” means that the specified group bears no substituents but the respective positions are occupied by hydrogen atoms only.

In some embodiments, X is —NH or —O—.

In some embodiments, R₁ is a substituted or unsubstituted C₁-C₁₀ alkyl.

In some embodiments, R₁ is ethyl or propyl.

In some embodiments, R₁ is —CH₂CH₂F.

In some embodiments, R₁ is —CH₂CCl₃.

In some embodiments, R₁ is a substituted or unsubstituted C₇-C₁₀ alkyl-aryl.

In some embodiments, R₁ is benzyl.

In some embodiments, R₂ is at the 3-position (meta) and OH is at the 2-position (ortho) of the phenyl ring.

In some embodiments, R₂ is a substituted or unsubstituted C₁-C₁₀ alkoxy.

In some embodiments, R₂ is ethoxy.

In preferred embodiments, the compound of Formula (I) is selected from the group consisting of:

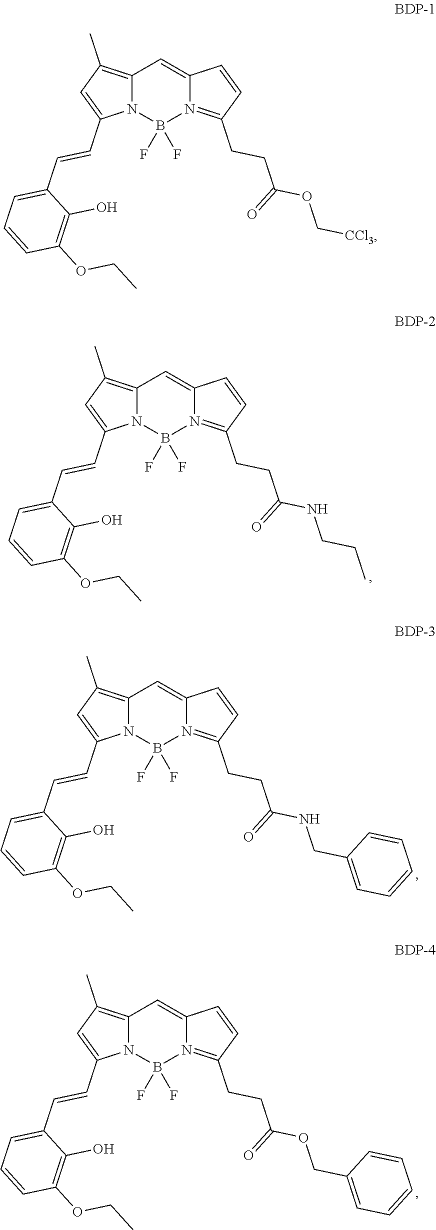

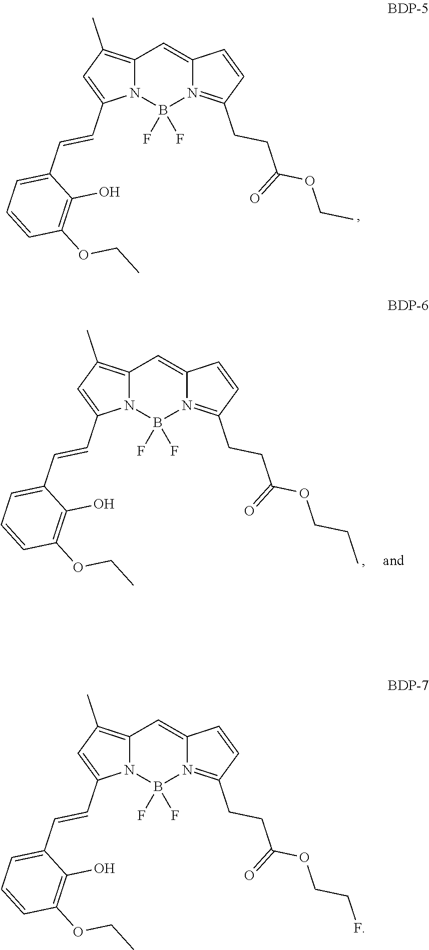

In accordance with the present invention, the compounds disclosed herein are fluorescent and can be detected using techniques well established in the art.

In a second aspect, the invention provides a conjugate comprising the compound of the invention and a detectable marker.

By “conjugate” is meant a compound of the invention covalently coupled to a detectable marker.

The term “detectable marker”, as used herein, refers to any agent that can produce a diagnostic signal detectable by any means in a subject.

The detectable marker of the invention may be a protein, nucleic acid molecule, compound, small molecule, organic compound, inorganic compound, or any other molecule with the desired properties suited for the practice of the present invention.

In some embodiments, the detectable marker according to the invention may be an imaging agent. The imaging agent can be any agent known to one of skill in the art to be useful for imaging, preferably being a medical imaging agent. Examples of medical imaging agent include, but are not limited to, single photon emission computed tomography (SPECT) agents, positron emission tomography (PET) agents, magnetic resonance imaging (MRI) agents, nuclear magnetic resonance imaging (NMR) agents, x-ray agents, optical agents (e.g., fluorophores, bioluminescent probes, near infared dyes, quantum dots), ultrasound agents and neutron capture therapy agents, computer assisted tomography agents, two photon fluorescence microscopy imaging agents, and multi-photon microscopy imaging agents, Exemplary detectable markers include radioisotopes (ie ¹⁸F, ¹¹C, ¹³N, ⁶⁴Cu, ¹²⁴I, ⁷⁶Br, ⁸²Rb, ⁶⁸Ga ^{99m}Tc, ¹¹¹In, ²⁰¹Tl or ¹⁵O, which are suitable for PET and/or SPECT use) and ultra-small superparamagnetic particles of iron oxide (USPIO) which are suitable for MRI.

It should be noted that the compound of the invention is fluorescent and thus may be used without a detectable marker. The detectable marker herein referred to is a further detectable marker distinct from the compound as such, and should not interfere with the binding of the compound.

The conjugates may be prepared using standard techniques known to those skilled in the art of synthetic organic chemistry, or may be deduced by reference to the pertinent literature.

In a third aspect, the invention provides a pharmaceutical composition comprising the compound or conjugate of the invention, and a pharmaceutically acceptable carrier.

The term “pharmaceutically acceptable” is employed herein to refer to those materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically-acceptable carrier” as used herein means a pharmaceutically-acceptable material, composition or vehicle, such as a liquid or solid filler, diluent, excipient, or solvent encapsulating material, involved in carrying or transporting the subject extract from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; sterile distilled water; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. See Remington: The Science and Practice of Pharmacy, 19th Ed. (Easton, Pa.: Mack Publishing Co., 1995), which discloses typical carriers and conventional methods of preparing pharmaceutical formulations.

Wetting agents, emulsifiers and lubricants, such as sodium lauryl sulfate and magnesium stearate, as well as coloring agents, release agents, coating agents, and perfuming agents, preservatives and antioxidants can also be present in the compositions.

Prior to being used in a specific application, pharmaceutical formulations composed of one or more of the compounds or conjugates of the invention in association with a pharmaceutically acceptable carrier may need to be formulated. Proper formulation is dependent upon the route of administration chosen.

In some embodiments, the pharmaceutical compositions of the present invention are formulated for administration in routes including, without limitation, depot, oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Depending on the intended mode of administration, the pharmaceutical formulation may be a solid, semi-solid or liquid, such as, for example, a tablet, a capsule, caplets, a liquid, a suspension, an emulsion, a suppository, granules, pellets, beads, a powder, or the like, preferably in unit dosage form suitable for single administration of a precise dosage. Suitable pharmaceutical compositions and dosage forms may be prepared using conventional methods known to those in the field of pharmaceutical formulation and described in the pertinent texts and literature, e.g., in Remington: The Science and Practice of Pharmacy, cited above.

In a fourth aspect, the invention provides a method for selectively detecting oligomeric proteins or peptides associated with a conformational disorder in a sample, the method comprising:

(a) contacting the sample with the compound or conjugate of the invention, wherein the compound binds selectively to the oligomers, under conditions that allow binding of the compound to the oligomers; and
(b) detecting the protein oligomers by measuring the signal of the complexes formed between the oligomers and said compound or conjugate.

A “conformational disorder”, as used herein, refers to any disorder whose pathogenesis is related to a structural change of a normal self protein or peptide into an oligomeric form with a high β-sheet content that is associated with toxicity.

In preferred embodiments, the conformational disorder is a neurodegenerative disorder. In the context of the present invention, the term “neurodegenerative disorder” refers to a disease or disorder selected from the group consisting of Alzheimer's disease, Parkinson's disease, Huntington's disease, frontotemporal lobar degeneration associated with protein TDP-43 (FTLD-TDP) (Types 1-4), Down syndrome, frontotemporal lobar degeneration associated with protein tau (FTLD-tau) (e.g., Pick's disease, corticobasal degeneration, progressive supranuclear palsy), a tauopathy, frontotemporal lobar degeneration associated with protein FUS (FTLD-FUS), Dementia with Lewy bodies (DLB), Amyotrophic lateral sclerosis (ALS), Mild Cognitive Impairment (MCI), prion diseases, British Dementia, Danish Dementia, HCHWA-D (hereditary cerebral haemorrhage with amyloidosis, Dutch type) and chronic tramatic encephalopathy (CTE).

Among them, prion diseases, including bovine spongiform encephalopathy (“mad cow disease”) and its human counterparts, e.g., Kuru, Creutzfeldt-Jakob disease, Gerstmann-Straussler-Scheinker disease, and fatal familial insomnia, are rare neurodegenerative disorders caused by an unusual type of infectious agent (prion) that consist of a self-propagating protein molecule. Prion diseases are caused by conversion of PrP^{c}, a normal cell-surface glycoprotein, into PrP^{Sc}, a conformationally altered isoform that serves as a molecular template for generation of additional molecules of PrP^{c}.

Many types of neurodegenerative disorders are linked with abnormal protein folding, accumulation, aggregation, and/or deposition of proteins or peptides, which are herein referred to as “proteins or peptides associated with a neurodegenerative disorder”. For example, there are two types of abnormal protein deposits in the brains of Alzheimer's patients. There are amyloid plaques composed of amyloid beta peptides that are deposited extracellularly in the brain parenchyma and around the cerebral vessel walls, and there are neurofibrillary tangles that are composed of aggregates of hyperphosphorylated tau protein located in the cytoplasm of degenerating neurons. In patients with Parkinson's Disease, Lewy bodies are observed in the cytoplasm of neurons of the substantia nigra. The major constituents of Lewy bodies are fragments of a protein named α-synuclein. In patients with Huntington's disease, intranuclear deposits of a polyglutamine-rich version of the mutant Huntingtin protein are a typical feature of the brain. Patients with hereditary Amyotrophic Lateral Sclerosis have aggregates primarily composed of TDP-43 (most commonly) or less frequently of superoxide dismutase in cell bodies and axons of motor neurons. Additionally, diverse forms of transmissible spongiform encephalopathy are characterized by accumulations of protease-resistant aggregates of the prion protein.

By “oligomeric proteins or peptides associated with a conformational disorder” is meant multimer species of protein or peptide monomers associated with a disorder that result from self-association of monomeric species.

Oligomeric proteins or peptides associated with a conformational disorder include, without limitation, beta amyloid, α-synuclein, prion, amylin, huntingtin, TAR DNA binding protein-43 (TDP-43), tau, A-Bri, A-Dan, and FUS RNA binding protein,

In preferred embodiments, the oligomeric proteins or peptides are Aβ oligomer or PrP^{Sc} oligomers.

The term “Aβ”, as used herein, refers to a family of peptides that are the principal chemical constituent of the senile plaques and vascular amyloid deposits (amyloid angiopathy) found in the brain in patients of Alzheimer's disease, Down's Syndrome, and Hereditary Cerebral Hemorrhage with Amyloidosis of the Dutch-Type (HCHWA-D). Amyloid β-Peptide (Aβ) is also known in the art as “amyloid beta protein,” “amyloid beta peptide,” “A beta,” “beta AP,” “A beta peptide,” or “Aβ peptide.” In whatever form, Aβ is a fragment of beta-amyloid precursor protein (APP). Aβ comprises variable number of amino acids, typically 39-43 amino acids. The term “Aβ” also refers to related polymorphic forms of Aβ, including those that result from mutations in the Aβ region of the APP normal gene.

Aβ oligomers may include a dynamic range of dimers, trimers, tetramers and higher-order species following aggregation of synthetic Aβ monomers in vitro or following isolation/extraction of Aβ species from human brain or body fluids. By “Aβ species” is meant an individual Aβ having a particular amino acid sequence. An Aβ species is commonly designated as “Aβ_{x-y}” wherein x represents the amino acid number of the amino terminus of the Aβ and y represents the amino acid number of the carboxy terminus. For example, Aβ₁₋₄₃ is an Aβ species or variant, whose amino terminus begin at amino acid number 1 and carboxy terminus ends at amino acid number 43. Examples of other Aβ species includes, but not limited to, (1) Aβ whose amino-terminus begin at amino acid number 1 of Aβ₁₋₄₃ shown above and whose carboxy-terminus ends at different amino acid number, such as Aβ₁₋₃₉, Aβ₁₋₄₀, Aβ₁₋₄₁, and A131.42, (2) Aβ whose amino acid sequences differ from Aβ₁₋₄₃ shown above at the amino-terminus or both termini, such as Aβ₃₋₄₀, Aβ₃₋₄₂, Aβ₄₋₄₂, Aβ₉₋₄₂ and Aβ₁₁₋₄₂.

In accordance with the present invention, the compounds of Formula (I) are fluorescent probes that preferentially form complexes with the oligomeric proteins or peptides over monomers or fibrils, with said complexes being then detectable via the fluorescence of the compounds, and therefore can be used for the selective detection of the oligomers in a sample. In some embodiments, the detectable markers coupled to the compounds disclosed herein may be used for the detection.

In another embodiment, the oligomeric protein or peptide detected using the methods, compounds and conjugates of the present invention are amyloidogenic proteins or peptides. As used herein, “amyloidogenic protein” or “amyloid protein” encompasses any protein/peptide aggregate that is associated with intra- or extracellul deposits within the body, with the most toxic aggregated forms being oligomeric. Amyloidogenic protein/peptide aggregation and deposition may be organ-specific (e.g., central nervous system, pancreas, etc.) or systemic. It should be noted that the disorders associated with protein or peptide oligomers are not limited to the central nervous system (CNS). In accordance with the invention, the oligomeric proteins or peptides subject to detection may result from monomeric beta protein precursor, prion proteins, α-synuclein, tau, ABri precursor protein, ADan precursor protein, amylin, apolipoprotein AI, apolipoprotein AII, lyzozyme, cystatin C, gelsolin protein, atrial natriuretic factor, calcitonin, keratoepithelin, lactoferrin, immunoglobulin light chains, transthyretin, A amyloidosis, β2-microglobulin, immunoglobulin heavy chains, fibrinogen alpha chains, prolactin, keratin, apolipoprotein CII, apolipoprotein E, amylin and medin.

In the context of the whole invention, the conformational disorders associated with protein or peptide oligomers include, without limitation, the neurodegenerative diseases disclosed supra (i.e., Alzheimer's disease, diffuse Lewy body disease, Down syndrome, frontotemporal dementia, Parkinson's disease, hereditary cerebral hemorrhage with amyloidosis, kuru, Creutzfeldt-Jakob disease, Gerstmann-Straussler-Scheinker disease, fatal familial insomnia, British familial dementia, Danish familial dementia), as well as familial corneal amyloidosis, Familial corneal dystrophies, medullary thyroid carcinoma, insulinoma, type 2 diabetes, isolated atrial amyloidosis, pituitary amyloidosis, aortic amyloidosis, plasma cell disorders, familial amyloidosis, senile cardiac amyloidosis, inflammation-associated amyloidosis, familial Mediterranean fever, dialysis-associated amyloidosis, systemic amyloidosis, and familial systemic amyloidosis.

The design of appropriate procedures for the detection of the oligomeric proteins or peptides associated with a conformational disorder is within the knowledge of the skilled person. “Selective”, as used herein, means that the compounds predominantly bind to oligomeric proteins or peptides, compared to other structurally closely related substances, namely monomers and fibrils of said proteins or peptides, and thus allows a distinction therebetween. Said selectivity may entail that the affinity for one of the species, such as the oligomers, is at least 2-fold, at least 3-fold, at least 4-fold, at least 5-fold, or >5-fold higher than that for other structurally closely related substances.

“Detecting”, as used herein, refers to determining the presence and, optionally, also the amount of a target substance of interest.

The term “sample” is defined by its ordinary meaning understood by a person skilled in the art and refers to any biological material containing or potentially containing one or more of the oligomeric proteins or peptides disclosed supra, in which the presence or amount of the oligomeric proteins or peptides can be determined using the methods and/or compounds and conjugates of the invention. The sample can be in any form such as fluids, solids, and tissues.

In certain embodiments, the sample contains a mixture of oligomeric proteins or peptide. For example, the sample may contain a mixture of any one or more beta amyloid, α-synuclein, prion, huntingtin, TAR DNA binding protein-43 (TDP-43), tau, A-Bri, A-Dan, and FUS RNA binding protein oligomers.

In another embodiment, the sample contains a mixture of oligomeric and non-oligomeric forms of the one or more aforementioned proteins or peptides thereof. For example, a sample may contain a mixture of Aβ oligomeric and non-oligomeric species, i.e., the sample may comprise a mixture of Aβ oligomers and Aβ monomers, a mixture of Aβ oligomers and Aβ fibrils, and/or a mixture of Aβ oligomers, Aβ monomers and Aβ fibrils. The term “Aβ fibrils” or “fibrils” as used herein refers to insoluble, fiber-like species of Aβ that can, for example, be detected in human and transgenic mouse brain tissue because of their birefringence with dyes such as Congo Red. Aβ species that form fiber-like structures as viewed by ultrastructural methods such as electron microscopy, comprised of stacks of Aβ monomers in β-pleated sheets. In such embodiments, the compounds or conjugates disclosed herein allow the selective detection of oligomers over monomers and fibrils of Aβ.

In a fifth aspect, the invention provides a method for diagnosing a conformational disorder associated with protein or peptide oligomers in a subject, comprising administering to said subject an effective amount of the compound or conjugate of the invention and measuring the signal of the complexes formed between said oligomers and said compound or conjugate.

By the term “effective amount” is meant a nontoxic but sufficient amount of the substance of the invention to provide the desired effect.

Monitoring disorders associated with protein or peptide oligomers disclosed supra in living patients and animals is limited by the availability of detection means. For example, a definite diagnosis of neurodegenerative disorders is usually only possible after brain tissue autopsy by monitoring number and distribution of plaques and tangles. Hence, developing means to identify plaques in vivo is essential for diagnosis as well as for evaluation of disease progression in response to therapies.

In accordance with the present invention, the compounds or conjugates of the invention are able to cross the blood-brain barrier in vivo and form complexes with protein or peptide oligomers associated with a neurodegenerative disease, and by measuring the fluorescence emitted by said compounds or more preferably the signal of the detectable markers coupled to the compounds, the presence of and/or risk of developing the neurodegenerative disease in a subject could be diagnosed. Similarly, for individuals having a neurodegenerative disease, the compounds and conjugates of the present invention are useful for monitoring the progression or regression of the disease with or without therapeutic intervention.

Examples of the neurodegenerative disorders suited for the diagnostic methods of the invention include, without limitation, any of the neurodegenerative diseases disclosed supra. In one embodiment, the neurodegenerative disease diagnosed using the methods and compounds of the present invention is Alzheimer's Disease. In another embodiment, the neurodegenerative disease diagnosed using the methods and compounds of the present invention is a prion disease.

The invention employs compounds or conjugates selective for oligomeric proteins or peptides associated with a conformational disorder which, in conjunction with non-invasive neuroimaging techniques such as magnetic resonance spectroscopy (MRS) or imaging (MRI), optical imaging, or gamma imaging such as positron emission tomography (PET) or single-photon emission computed tomography (SPECT), are used to quantify said oligomeric proteins or peptides in vivo. Other in vivo imaging techniques that can be employed in the methods of the present invention include, without limitation, near infra-red imaging, computer assisted tomography, two photon fluorescence microscopy imaging, and multi-photon microscopy imaging

For purposes of in vivo imaging, the type of detection instrument available is a major factor in selecting a certain detectable marker. For instance, radioactive isotopes and ¹⁸F are particularly suitable for in vivo imaging in the methods of the present invention. The type of instrument used will guide the selection of the radionuclide or stable isotope. For instance, the radionuclide chosen must have a type of decay detectable by a given type of instrument. Another consideration relates to the half-life of the radionuclide. The half-life should be long enough so that it is still detectable at the time of maximum uptake by the target, but short enough so that the host does not sustain deleterious radiation. The radiolabeled compounds of the invention can be detected using gamma imaging wherein emitted gamma irradiation of the appropriate wavelength is detected. Methods of gamma imaging include, but are not limited to, SPECT and PET. Preferably, for SPECT detection, the chosen detectable marker will lack a particulate emission, but will produce a large number of photons in a 140-200 keV range. For PET detection, the radiolabel will be a positron-emitting radionuclide such as ¹⁸F which will annihilate to form two 511 keV gamma rays which will be detected by the PET camera.

The methods of the present invention may use isotopes detectable by any of the methods described supra. Suitable radioisotopes for purposes of this invention include beta-emitters, gamma-emitters, positron-emitters, and x-ray emitters. Examples of detectable isotopes include, without limitation, ¹⁸F, ¹⁹F, ¹²³I, ⁿC ²H, ¹¹C, ¹³C, ¹⁴C, ¹⁸C, ¹³N, ¹⁵N, ¹⁵0 ¹⁷0, ¹⁸0, ¹⁸F, ³⁵S, ³⁶Cl, ⁷⁵Br, ⁷⁶Br, ⁷⁷Br, ⁸²Br, ¹²⁰I, ¹²³I, ¹²⁴I, ¹²⁵I, ¹³¹I, ⁶⁷Ga, ^{81m}Kr, ⁸²Rb, ^{m}In, ¹³³Xe, ²⁰¹Tl, ⁹⁰Y or ^{99m}Tc. Suitable stable isotopes for use in Magnetic Resonance Imaging (MRI) or Spectroscopy (MRS), according to this invention, include ¹⁹F and ¹³C. Suitable radioisotopes for in vitro quantification of amyloid in homogenates of biopsy or post-mortem tissue include ¹²⁵I, ¹⁴C, and ³H. The preferred radiolabels are ¹⁸F for use in PET in vivo imaging, ¹²³I for use in SPECT imaging, ¹⁹F for MRS/MRI, and ³H or ¹⁴C for in vitro studies. However, any conventional method for visualizing diagnostic probes can be utilized in accordance with this invention.

In another embodiment, the detectable label comprises a microparticle or a nanoparticle, such as a gold particle, a magnetic, supramagnetic or ferromagnetic particle, a lanthanide particle (e.g. Gd, Eu or Nd) optionally doped with metal, or a nanocrystal (such as a quantum dot). What specific label is used will vary with the used imaging method and may be chosen by the skilled person.

Administration to the subject may be local or systemic and accomplished intravenously, intraarterially, intrathecally (via the spinal fluid) or the like. Administration may also be intradermal or intracavitary, depending upon the body site under examination. After a sufficient time has elapsed for the compound or conjugate to bind to the oligomeric proteins or peptides, for example 30 minutes to 48 hours, the area of the subject under investigation is examined by routine imaging techniques such as those described supra, for example, and without limitation, MRS/MRI, SPECT, planar scintillation imaging, and PET. Alternative and emerging imaging techniques such as Matrix-Assisted Laser Desorption Ionization (MALDI) imaging mass spectrometry, can also be employed. The exact protocol will necessarily vary depending upon factors specific to the patient, and depending upon the body site under examination, method of administration and type of label used; the determination of specific procedures would be routine to the skilled artisan.

In a final aspect, the invention provides a method for treating or preventing a conformational disorder associated with protein or peptide oligomers in a subject, comprising administering to said subject an effective amount of the compound or pharmaceutical composition of the invention.

The terms “treating” and “treatment”, as used herein, refer to reduction in severity or frequency of symptoms, elimination of symptoms or underlying cause, prevention of the occurrence of symptoms or their underlying cause, and improvement or remediation of damage. As used herein, treating and treatment also include prophylactic treatment, i.e., the prevention of, inhibition of, slowing of, or amelioration of, the possible onset or onset of a condition.

According to the invention, the subject of interest is a mammal, preferably a human, and may also be, for diagnostic purposes, a human suspected of having a disorder associated with protein or peptide oligomers.

In various embodiments, the disorder is a neurodegenerative disorder.

In preferred embodiments of the present invention, the neurodegenerative disease is any one of the neurodegenerative diseases disclosed supra. In one embodiment, the neurodegenerative disease is Alzheimer's Disease (AD). In another embodiment, the neurodegenerative disease is a prion disease.

The present invention is further illustrated by the following examples. However, it should be understood, that the invention is not limited to the exemplified embodiments.

### EXAMPLES

### Materials and Methods

### Reagents and Solvents

The chemicals, including aldehydes and solvents, were purchased from Sigma Aldrich, Fluka, MERCK, Acros and Alfa Aesar. All the chemicals were directly used without further purification. Normal phase column chromatography purification was carried out using MERCK silica Gel 60 (Particle size: 230-400 mesh, 0.040-0.063 mm).

### Diversity-Oriented Fluorescence Library (DOFL) High-Throughput/Content Screening

DOFL compounds were diluted from 1 mM DMSO stock solutions with the culture medium to make final concentration of 1 μM. Chinese Hamster Ovary (CHO) cells and 7PA2 cells, which were both kindly donated by Dr. Edward H. Koo (University of California, San Diego), were plated side by side in 384-well plates and incubated with DOFL compounds for 2 h at 37° C. 7PA2 cells were stably transfected with plasmid encoding APP751 with V717F mutation and were reported to produce low MW Aβ oligomers (up to 4-mer) in intracellular vesicles prior to secretion into the cell culture medium (Walsh, D. M., et al. Nature 2002, 416, 535). Detailed characterization of 7PA2 cells has been reported in the literature (Podlisny, M. B., et al. J. Biol. Chem. 1995, 270, 9564; Podlisny, M. B., et al. J. Biochemistry 1998, 37, 3602). The fluorescence cell images of two regions per well were acquired using ImageXpress Micro™ cellular imaging system (Molecular Device, Sunnyvale, Calif.) with 10× objective lens and the intensity was analyzed by MetaXpress® image processing software (Molecular Devices, Sunnyvale, Calif.) and by manual observation. The compounds preferably staining 7PA2 cells over CHO cells were selected as candidates.

### Peptide Preparation

Synthetic Aβ1-40 was purchased from American Peptide Co. (Sunnyvale, Calif.) in lyophilized form. Dry peptide was dissolved in 1,1,1,3,3,3-hexafluoro-2-isopropanol (HFIP) and incubated at 25° C. for 1 h to remove any preformed aggregates. It was aliquoted into small aliquots and dried using a speed-vac. The dry peptide was stored at −20° C. until required, where each aliquot was then dissolved in 5 M GuHCl 10 mM Tris.Cl pH 8 to 1 mM peptide solution. After sonication in a sonicating water bath for 15 min, the solution is diluted with phosphate buffered saline (PBS), pH 7.4 and stored on ice until use. This freshly prepared sample is referred to as monomer (Ryan, T. M., et al. J. Biol. Chem. 2012, 287, 16947). To form fibrils, 100 μM of the sample is incubated for 24 h at 37° C. with 5 s shaking at every 7 min interval. Pre-formed oligomers were prepared by Aβ1-40 peptide solubilized in borate buffered saline (50 mM BBS/PBS) and reacted with 5 mM glutaraldehyde overnight at 37° C. to produce stable oligomers by controlled polymerization, as previously described (Goni, F., et al. PloS one 2010, 5; Goni, F., et al. J. Neuroinflammation 2013, 10, 150). The solution was neutralized with Tris buffer then dialyzed against deionized distilled water overnight and lyophilized. Prior to fluorescence assays, it is re-solubilized in deionized distilled water and diluted in PBS. Western blot performed on the sample with anti-Aβ 4G8/6E10 as primary antibody, revealed major band of about 80 kDa and higher without monomers. By electron microscopy, the sample makes spheres of 10-20 nm.

### Time-Dependent Fibril Formation

For monitoring of fibril formation over time, 40 μM peptide solution of Aβ1-40 was prepared as above and incubated at 37° C. with 5 s shaking at every 7 min interval. Fluorescence readings were taken at various time point intervals by mixing 30 μL aliquot of peptide solution to 10 μM of dye. ThT signal was monitored at 480 nm by 444 nm excitation, whereas BDP-1 was excited at 530 nm and its emission detected at 585 nm. Fluorescence was measured using SpectraMax M2 spectrophotometer (Molecular Devices, Sunnyvale, Calif.). Aβ1-40 was also co-incubated with dye to study any effects the dye may have on fibril formation.

### Dot Blot Analysis

3 μL of 40 μM Aβ1-40 sample were spotted onto nitrocellulose membrane (Bio-Rad) at selected time points. The membranes were blocked by 10% (w/v) fat-free milk in 50 mM Tris 150 mM NaCl, pH 7.4 and 0.05% (v/v) Tween-20 (TBST buffer) for 1 h at room temperature, followed by incubation with anti-oligomer polyclonal A11 antibody (1:1000 dilution; Invitrogen) or anti-Aβ1-16 (6E10) monoclonal antibody (1:1000 dilution; Covance) in 5% (w/v) fat-free milk and TBST buffer overnight at 4° C. The membranes were washed 3 times in TB ST before incubation with anti-rabbit or anti-mouse antibody (1:5000 dilution) in 5% (w/v) fat-free milk and TBST buffer at room temperature for 1 h.

### Pelleting Assay

Aβ1-40 samples were incubated at 37° C. At selected time points, aliquots of 150 μL were removed and subjected to centrifugation at 100,000 rpm (TL-100 rotor, Beckman) for 20 min at 4° C. Under these centrifugation conditions, monomeric Aβ does not sediment significantly. The concentration of monomeric Aβ in the supernatant after centrifugation was monitored using fluorescence measurements based on the reaction of fluorescamine with primary amine groups. The supernatants (45 μL) were added to a microtitre plate along with 15 μL of 1 mg/mL fluorescamine in DMSO. Samples were incubated at room temperature for 5 min and fluorescence intensities were measured using SpectraMax M2 spectrophotometer (Molecular Devices, Sunnyvale, Calif.) with excitation and emission filters of 355 nm and 460 nm, respectively.

### Transmission Electron Microscopy

At selected time points, Aβ1-40 sample incubated at 37° C. was removed and applied to freshly glow-discharged carbon-coated copper grids. The grids were then stained with several drops of 2% potassium phosphotungstate, pH 6.8, and examined using an FEI Tecnai 12 transmission electron microscope operating at 120 kV. Images were obtained using an Olympus SiS MegaViewIII charge-coupled device camera.

### Ex Vivo Imaging of Brains

For ex vivo imaging, a stock solution of BDP-1 was made at 10 mM in 100% DMSO. 18 month old APP/PS1 transgenic (Tg) AD model mice were given intraperitoneal (I.P.) injections with either 1.25 μL BDP-1 diluted in 500 μL saline (n=2) or 500 μL saline alone (n=2). APP/PS1 Tg mice develop amyloid plaques from 4 months of age (Holcomb, L., et al. Nat. Med. 1998, 4, 97). Mice were anesthetized with an overdose of sodium pentobarbital and perfused 0.1 M PBS, pH7.4. Brains were removed 24 h after the I.P. injection and fixed by immersion in periodate-lysine-paraformaldehyde for 24 h, cryo-protected in 30% sucrose for 3 days and sectioned into 40 μm coronal sections using a cryostat. Brain sections from the BDP-1 injected mouse and the control APP/PS1 mouse that received a saline alone injection were then stained for Aβ using fluorescent immunohistochemistry. Briefly, free floating sections were incubated with MOM blocking reagent (Vector) followed by an overnight incubation at 4° C. with anti-Aβ antibodies 4G8 and 6E10 diluted in MOM protein concentrate (Vector), as previously published (Goni, F., et al. J. Neuroinflammation 2013, 10, 150; Scholtzova, H., et al. Acta Neuropathol. Commun. 2014, 2, 101). Sections were then incubated with a 488 conjugated secondary antibody (Jackson Immunoresearch) for 2 h at room temperature, mounted onto slides and cover slipped. Staining was visualized using a LMD6500 fluorescent microscope (Leica); 6E10/4G8 staining was imaged using in the green (488) channel and BDP-1 was imaged in the red (561) channel.

### Computational Details

Geometry of BDP-1 was quantum mechanically optimized in a gas phase as well as in an aqueous phase. The stable complex structure of BDP-1 with Aβ oligomer was executed by molecular docking search followed by all-atom, explicit water molecular dynamics simulations. Thermodynamic analysis was then performed by applying the liquid integral-equation theory to simulated complex conformations. Further details are provided as below.

### Measurements and Analysis

HPLC-MS was taken on an Agilent-1200 with a DAD detector and a single quadrupole mass spectrometer (6130 series). The analytical method, unless indicated, is A: H₂O (0.1% HCOOH), B: CH₃CN (0.1% HCOOH), gradient from 10 to 90% B in 10 minutes; C18 (2) Luna column (4.6×50 mm2, 3.5 μm particle size).

Spectroscopic and quantum yield data were measured on a SpectraMax M2 spectrophotometer (Molecular Devices). Compounds in solvent (100 μL) in 96-well polypropylene plates was for fluorescence measurement. Data analysis was performed using Graph Prism 5.0.

¹H-NMR and ¹³C-NMR spectra were recorded on Bruker AMX500 (500 MHz) spectrometers, and chemical shifts are expressed in parts per million (ppm) and coupling constants are reported as a J value in Hertz (Hz).

### Quantum Yield Measurements

Quantum yields for BDP-1 were measured by dividing the integrated emission area of their fluorescent spectrum against the area of Rhodamine B in EtOH excited at 490 nm (Φ_{rho-B}=0.7) (Arbeloa, F. L., et al. J. Lumin. 1989, 44, 105). Quantum yields were then calculated using equation (1), where F represents the integrated emission area of fluorescent spectrum, η represents the refractive index of the solvent, and Abs represents absorbance at excitation wavelength selected for standards and samples. Emission was integrated from 530 nm to 750 nm.

$${Φ}_{flu}^{sample} = {Φ}_{flu}^{reference} ( \frac{{F}^{sample}}{{F}^{reference}} ) ( \frac{{η}^{ sample}}{{η}^{ reference}} ) ( \frac{{Abs}^{reference}}{{Abs}^{sample}} ) \qquad ( 1 )$$

### CD Spectroscopy

CD measurements were made using an Aviv model 62 DS CD spectrometer (Aviv Associates Inc., Lakewood, N.J.) at 25° C. with a 1-mm path length quartz cuvette, a spectral bandwidth of 1 nm, a signal averaging time of 1 s, and a data interval of 0.5 nm. The spectra presented are the averages of five measurements and corrected using a reference solution lacking Aβ.

### Quantum Mechanical Calculations

The geometry optimization for BDP-1 compound was performed by using density functional theory at the B3LYP/6-31G* level (Becke, A. D. J. Chem. Phys. 1993, 98, 1372) at the gas phase as well as an aquaous phase using Gaussian 09 program (Frisch M. J. et al., Gaussian 09 (Gaussian inc., Wallingford Conn., 2009)). Vibrational frequency analyses were executed to verify the identity of each stationary point as an energy minimum.

### Molecular Docking Search and Molecular Dynamics (MD) Simulations

BDP-1 docking search with Aβ oligomer were executed by using AutoDock 4.0 software package (Goodsell, D. S. and Olson, A. J. Proteins 1990, 8, 195). The docking simulations were carried out with a box centered on the Aβ oligomer and employing 50×50×50 grid points. For the Aβ oligomer structure, we used X-ray (4NTR) determined Aβ trimers derived from the β-amyloid peptide as a working model for toxic Aβ oligomer associated with Alzheimer's Disease (Spencer, R. K., et al. J. Am. Chem. Soc. 2014, 136, 5595). We used the Lennard-Jones (U) parameter of carbon for boron atom due to the absent of LJ parameter for boron. This is not a harsh substitution since boron atom has four coordination number in BDP-1 (Shi, X. G., et al. J. Phys. Chem. B 2008, 112, 12801; Iavarone, A. T., et al. J. Am. Chem. Soc. 2007, 129, 6726). Based on the global docking search, the most energy-minimized complex structure of BDP-1 with Aβ oligomer was used as an initial structure for MD simulations. We performed all-atom, explicit-water MD simulations using AMBER 14 package (Case D. A., et al. AMBER14 (University of California, San Francisco, 2014)) with the ff99SB force field (Hornak, V., et al. Proteins 2006, 65, 712) for the Aβ complex and the TIP4P-Ew model (Horn, H. W., et al. J. Chem. Phys. 2004, 120, 9665) for water. The 5,329 water molecules were added to the simulation box. The particle mesh Ewald (PME) method (Darden, T., et al. J. Chem. Phys. 1993, 98, 10089) was applied for dealing long-range electrostatic interactions while 10 Å cutoff was used for the short-range non-bonded interactions. The system was initially subjected to 500 steps of steepest descent minimization followed by 500 steps of conjugate gradient minimization while the complex structure was constrained by 500 kcal/(mol·Å²) harmonic potential. Then, the system was minimized using 1,000 steps of steepest descent minimization followed by 1,500 steps of conjugate gradient minimization without harmonic restraints. The system was subsequently subjected to a 20 ps equilibration process in which the temperature was gradually raised from T=0 to 310 K with a constant volume. This was followed by a 200 ps constant-pressure (NPT) ensemble simulation at T=310 K and P=1 bar. A 2 ns production run was then carried out at T=310 K and P=1 bar.

### Thermodynamics Calculations

The three-dimensional reference interaction site model (3D-RISM) theory was used (Hirata, F., Molecular Theory of Solvation (Kluwer, Dordrecht, 2003); Imai, T., et al. J. Chem. Phys. 2006, 125) to compute the solvation free energy ΔGₛₒₗᵥ of the BDP-1 complex with Aβ oligomer structure. This theory provides the equilibrium water distribution function around a given protein structure, with which ΔGₛₒₗᵥ can be computed by using the Kirkwood charging formula (Ben-Naim, A. Molecular Theory of Solutions (Oxford University Press, New York, 2006)). The internal energy (Eu) was directly computed from the force field used for the simulations. By combining the internal energy and the solvation free energy, a binding free energy (f=Eu+Gsolv) was obtained. To obtain a residue-specific contribution to the binding free energy, an exact decomposition method (Chong, S. H. and Ham, S. Chem. Phys. Lett. 2011, 504, 225) which provides the site-directed thermodynamic contributions to the free energy upon complexation was used. In FIG. 9, each bar represents the free energy difference (Δf) for each residue obtained from the free energy of Aβ oligomer with BDP-1 (fcomplex) relative to Aβ oligomer without BDP-1 (fAβ oligomer).

### Synthesis and Characterization

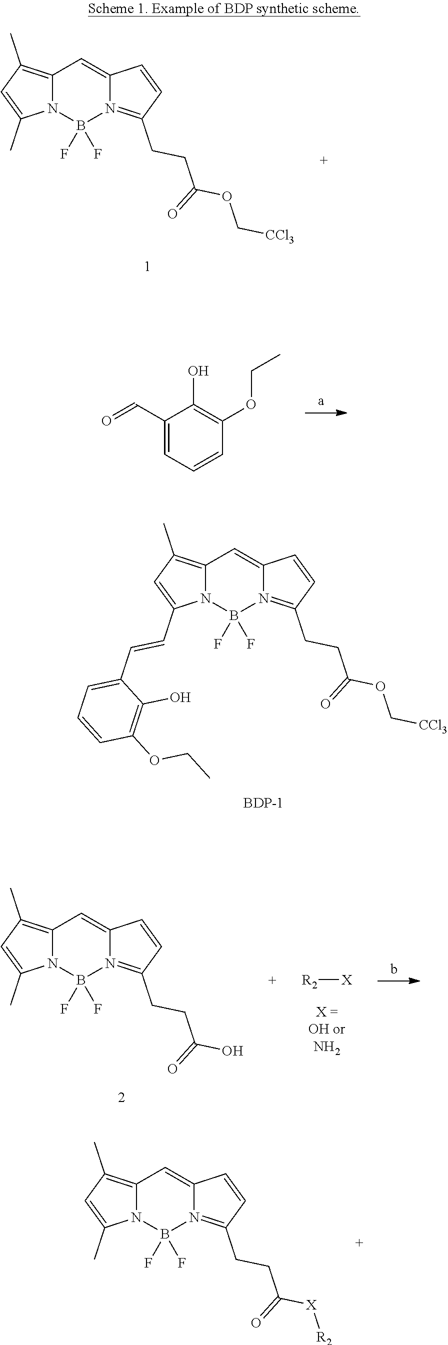

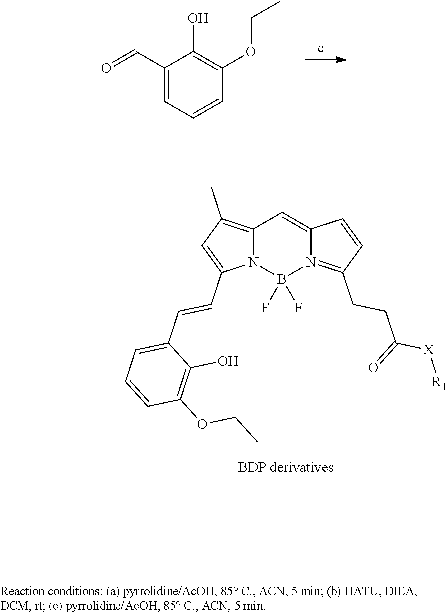

General procedure for BDP-SM synthesis: to an ice cold solution of compound 2 (0.07 mmol) in dry dimethylformamide (DMF) was added pyridine (0.34 mmol), followed by HATU (0.08 mmol) and stirred for 5 min. To the reaction mixture, corresponding alcohol/amine (0.34 mmol) was added and stirred overnight under room temperature. The reaction mixture was diluted with DCM (50 mL) and washed with water three times to remove the DMF. After removal of the DMF, the DCM part was dried over anhydrous Na₂SO₄, evaporated of the DCM to yield the crude compound. The crude compound was finally purified by silica gel chromatography in 7:3 hexane and ethyl acetate mixture.

General procedure for BDP synthesis: compound 1 or BDP-SM (20 mg, 47 μmol) and aldehyde (94 μmol, 2 equiv) were dissolved in acetonitrile (3 mL), with 6 equiv. of pyrrolidine (23.5 μL, 282 μmol) and 6 equiv. of AcOH (16.1 μL, 282 μmol). The condensation reaction was performed by heating to 90° C. for 5 min. The reaction mixture was cooled down to room and concentrated under vacuum, and purified by short silica column.

Procedure for BDP-1 synthesis: compound 1 (20 mg, 47 μmol) and aldehyde (94 μmol, 2 equiv) were dissolved in acetonitrile (3 mL), with 6 equiv. of pyrrolidine (23.5 μL, 282 μmol) and 6 equiv. of AcOH (16.1 μL, 282 μmol). The condensation reaction was performed by heating to 90° C. for 5 min. The reaction mixture was cooled down to room and concentrated under vacuum, and purified by short silica column (EtOAc/Hexane=2:3). Yield: 17.1 mg (63.8%).

Characterization of BDP-1: ¹H NMR (500 MHz, CDCl3) δ=7.70 (s, 2H), 7.28 (dd, J=7.6 Hz, 1.0, 1H), 7.02 (s, 1H), 6.82 (m, 4H), 6.28 (d, J=3.9 Hz, 1H), 4.78 (s, 2H), 4.20-4.04 (m, 2H), 3.39 (t, J=7.5 Hz, 2H), 2.96 (t, J=7.5 Hz, 2H), 2.25 (s, 3H), 1.45 (t, J=7.0 Hz, 3H); ¹³C NMR (126 MHz, CDCl₃): 171.05, 157.99, 155.12, 145.96, 144.73, 143.09, 136.88, 133.60, 133.52, 126.81, 122.40, 121.88, 119.73, 119.43, 118.84, 116.97, 116.29, 112.13, 94.89, 74.02, 64.72, 33.03, 23.68, 14.81, 11.30.

HRMS m/z (C₂₅H₂₄BCl₃F₂N₂O₄) calculated: 570.0863, found: 593.0775 (M+Na)⁺.

### Example 1: Oligomer-Specific Sensor Discovery (BDP-1) and Characterization

Since the proposed role of Aβ oligomers in the pathophysiology of AD, synthetic Aβ oligomers have been used as tools for the development of therapeutics and biomarkers. To develop Aβ oligomer-selective probe in living system, 7PA2 cells enriched in Aβ oligomers (Walsh, D. M. and Selkoe, D. J. J. Neurochem. 2007, 101, 1172) were incubated with 3,500 DOFL compounds (Im, C. N., et al. Angew. Chem. Int. Ed. Engl. 2010, 49, 7497; Kang, N. Y., et al. Angew. Chem. Int. Ed. Engl. 2013, 52, 8557; Yun, S. W., et al. Acc Chem. Res. 2014, 47, 1277). When in the absence of mechanistic cues to rationally design probes for Aβ oligomers, high-throughput screening was envisioned to be crucial in helping identify promising leads. By expanding this strategy, 5 candidate compounds were selected based on their higher fluorescence intensity in 7PA2 cells than in CHO cells, from which the 7PA2 cells were propagated. These candidates were narrowed by a more direct approach, using synthetically stabilized oligomer of Aβ in comparison to monomer and fibrils. While Aβ monomers and fibrils have been used widely, Aβ oligomer is challenging to form or maintain due to its dynamic nature. Aβ₁₋₄₀ peptide was solubilized in borate buffered saline (50 mM BBS/PBS) and reacted with 5 mM glutaraldehyde overnight at 37° C. to produce covalently stabilized Aβ oligomers, as previously described (Goni, F., et al. PloS one 2010, 5; Goni, F., et al. J. Neuroinflammation 2013, 10, 150). The most selective oligomer fluorescence turn-on probe was dubbed BoDipy-Oligomer or BDP-1 for short. With BDP-1, the highest fluorescence enhancement was observed upon incubation with Aβ oligomers indicating a preference for these intermediary conformations of Aβ aggregation over monomers or fibrils (FIG. 1).

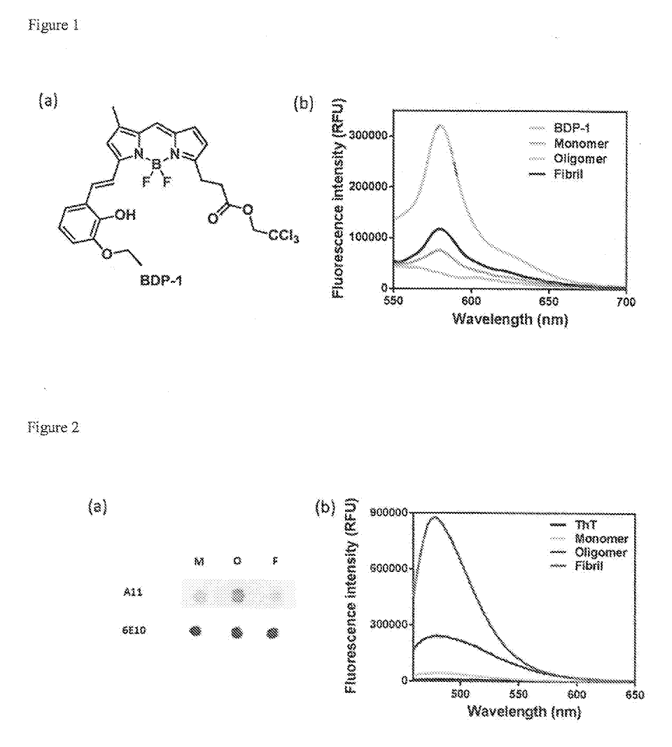

The conformations of different Aβ peptide preparation were confirmed by dot blot assays and the results showed that the oligomer responded most strongly to the anti-oligomer antibody (A11), which has been reported to specifically recognize a generic epitope common to pre-fibrillar oligomers but not monomers or fibrils (Kayed, R., et al. Science 2003, 300, 486) (FIG. 2a). Blotting a replicate membrane with anti-Aβ₁₋₁₆ (6E10) antibody, which does not discriminate different conformations of Aβ, showed similar amount of protein in all 3 Aβ preparations. Amyloid fibrils probe, ThT showed fluorescence response in the increasing order of freshly prepared Aβ monomers, followed by oligomer and fibrils as expected (FIG. 2b).

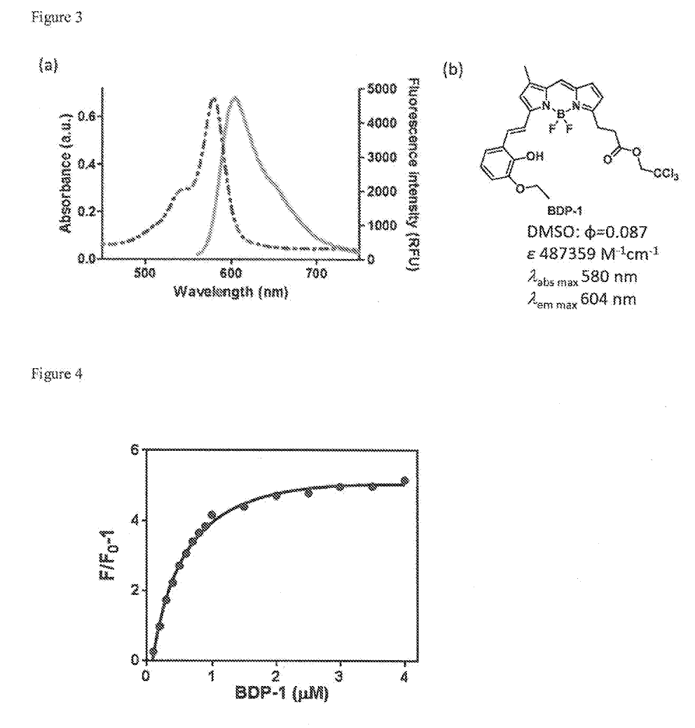

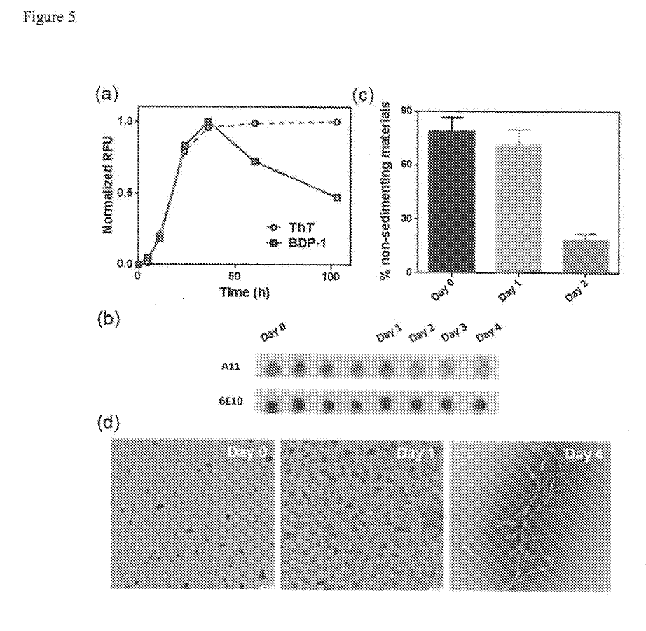

Photophysical properties of BDP-1 are characterized and summarized in FIG. 3. To quantify the affinity of BDP-1 for Aβ oligomers, the apparent binding constant (K_{d}) of BDP-1 was measured by conducting a saturation assay. Transformation of the saturation binding data to Scatchard plot, indicated affinity of BDP-1 for oligomers at a K_{d} value of 0.48 μM (FIG. 4).

TABLE 1
detailed statistics of FIG. 4.

| Best-fit values | |
|---|---|
| Bmax | 7.886 |
| Kd | 0.4819 |
| NS | −0.2092 |
| Background | −1.172 |
| Std. Error | |
| Bmax | 0.3667 |
| Kd | 0.07957 |
| NS | 0.09903 |
| Background | 0.2375 |
| 95% Confidence Intervals | |
| Bmax | 7.088 to 8.685 |
| Kd | 0.3085 to 0.6553 |
| NS | −0.4250 to 0.006613 |
| Background | −1.690 to −0.6546 |
| Goodness of Fit | |
| Degrees of Freedom | 12 |
| R2 | 0.9958 |
| Absolute Sum of Squares | 0.1423 |
| Sy.x | 0.1089 |
| Number of points Analyzed | 16 |

### Example 2: BDP-1 Detects Oligomers on Fibril Formation Pathway

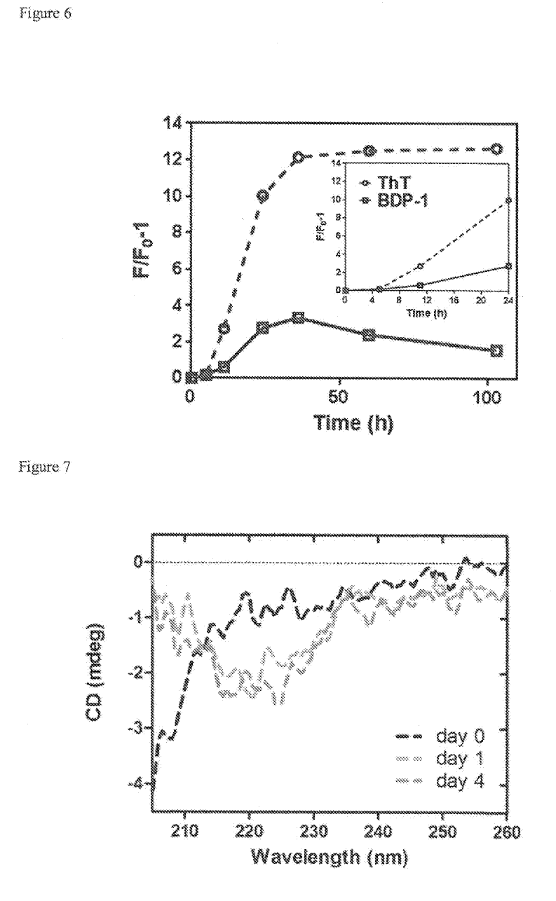

Next, the oligomer-sensing ability of BDP-1 over the course of Aβ fibril formation was investigated using the same peptide preparation, instead of 3 different pre-prepared conformations as described earlier. To do this, Aβ peptide was subjected to fibril forming conditions, and at each selected time point, a small aliquot was sampled and added to BDP-1 for fluorescence measurement. Concurrently, Aβ fibril formation samples were monitored with ThT, which reached a maximum fluorescence after about 1-day and plateaus for the remaining incubation period. Measurements with BDP-1 observed a gradual increase in fluorescence, which reached maximum fluorescence intensity at about day-1 incubation, followed by a decrease in signal over the remaining incubation period (FIG. 5a, FIG. 6). Fluorescence measurement of BDP-1 alone in the same manner revealed no change in its signal intensity (data not shown). Without wishing to be bound to any particular theory, it was postulated that the observed changes in fluorescence signal were an indication of BDP-1 detecting Aβ oligomeric species on-fibril pathway, whereby the signal increased as monomers aggregated into oligomers, but decreased as more Aβ assembled into fibrils.

To elucidate the aggregated species or the changes in protein conformations that BDP-1 may be recognizing, biophysical characterizations of the sample during Aβ fibril formation were performed. Particular attention was paid towards the day-1 species, where the probe had been observed to yield maximum fluorescence enhancement. Dot blots over the course of fibril formation showed that A11 recognized earlier species in the incubation, most intense at 3-5 h, as compared to BDP-1, which recognized the later (day-1) species (FIG. 5b). Pelleting assay showed that at day-1, quite similar to day-0, majority of Aβ were still in solution and had not aggregated into large sedimenting materials. This implied that the aggregated species which enhanced the fluorescence of BDP-1 were soluble, which was in stark contrast to the decrease in the fraction of soluble protein after 2-days incubation (FIG. 5c). At the same time, transmission electron microscopic (TEM) images taken at the end of the 4-days incubation confirmed the presence of Aβ fibrils. In contrast, TEM images captured either immediately after fibril formation had been initiated (day-0) or after 1-day incubation did not yet show any signs of fibrils (FIG. 5d). The secondary structure of Aβ analyzed by circular dichroism (CD) spectroscopy at selected time points indicated that Aβ was random coil when freshly initiated to form fibrils (day-0), consistent with reports in the literature (Ryan, T. M., et al. J. Biol. Chem. 2012, 287, 16947), while day-1 species was observed to possess β-sheet content, similar to fibrils formed at day-4 (FIG. 7). Taken together, the presence of β-sheet structure alone does not suffice to explain the binding specificity of the probe.

### Example 3: Structural Characteristics of AD Oligomer Complex with BDP-1

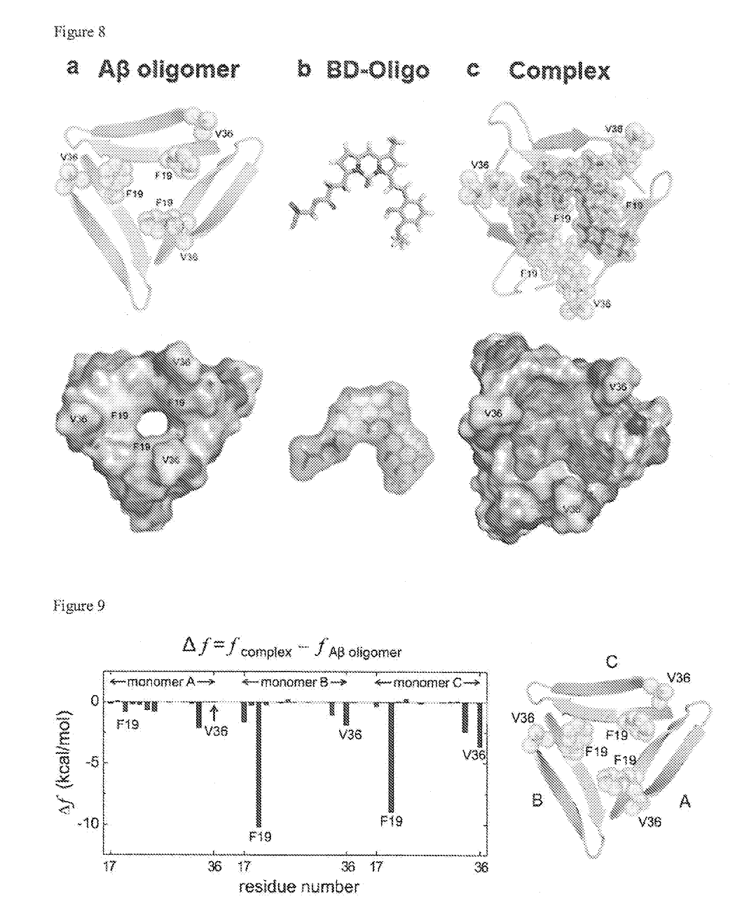

To understand the structural features and the binding specificity of BDP-1 for Aβ oligomer over Aβ monomer and fibrils, quantum mechanical calculations for BDP-1 were performed followed by molecular docking search and molecular dynamics (MD) simulations for the complex of BDP-1 and Aβ oligomer. To construct Aβ oligomer structure, X-ray determined Aβ trimers derived from the β-amyloid peptide were used as a working model for toxic Aβ oligomer associated with neurodegeneration in AD (FIG. 8a) (Spencer, R. K., et al. J. Am. Chem. Soc. 2014, 136, 5595). Though not a true depiction of structure, the described computation methods offer a possible approximation as starting point. BDP-1 is most stable as a planar form in gas phase, as well as in an aqueous environment based on quantum mechanical calculations at the B3LYP/6-31G* level (FIG. 8b). To search for the stable complex structure of BDP-1 with Aβ oligomer, molecular docking search was performed followed by all-atom, explicit water MD simulations (see the above-detailed computational methods). Upon complexation, BDP-1 adopted a conformational transition from planar to twisted geometry in order to maximize the interaction with Aβ oligomer (FIG. 8c). The main binding mode was pi-pi stacking interactions between the aromatic rings of BDP-1 and the exposed hydrophobic patches of Aβ oligomer. More specifically, the BODIPY ring and the phenyl ring of BDP-1 were recognized by hydrophobic F19/V36 residues in Aβ oligomer. Moreover, the carbonyl group of BDP-1 forms CH—O bonding with V36 side chain. These binding modes between BDP-1 and F19/V36 residues of Aβ oligomer were oligomer-specific, since F19/V36 residues were exposed to solvent only in Aβ oligomer, but not in Aβ fibrils (Luhr, T., et al. Proc. Natl. Acad. Sci. USA 2005, 102, 17342). In addition, the F19/V36 residues were also less exposed to solvents in Aβ monomer, which displayed intrinsically disorder in aqueous environments (Lee, C. and Ham, S. J. Comput. Chem. 2011, 32, 349). The exposed F19/V36 residues which are only present in Aβ oligomer and not (or much less) in Aβ fibril (Aβ monomer) are quite suitable for BDP-1 recognition by executing pi-pi stacking interactions, as well as H-bonding between them. This structural analysis offers the molecular motif on why BDP-1 is an Aβ oligomer-specific detector.

### Example 4: Thermodynamic Calculations for BDP-1 Complex with AD Oligomer

To further characterize the molecular origin and the binding affinity upon complexation of BDP-1 with Aβ oligomer, the changes in total internal energy (ΔEu), solvation free energy (ΔGₛₒₗᵥ), and free energy (Δf) upon its complexation were computed. The internal energy was directly computed from the force field used for the simulations, whereas the solvation free energy was calculated using the integral-equation theory of liquids (Imai, T., et al. J. Chem. Phys. 2006, 125, 024911). By combining the internal energy and the solvation free energy, a free energy (f=Eu+Gₛₒₗᵥ) was performed. The binding free energy upon BDP-1 complexation with Aβ oligomer was computed to be −27.2 kcal/mol in aqueous environments. Based on the site-directed thermodynamics analysis (Chong, S. H. and Ham, S. J. Chem. Phys. 2011, 135, 034506) of the binding free energy, it was evident that the hydrophobic residues of F19/V36 in Aβ oligomer contributed most distinctively to the binding free energy upon complexation (FIG. 9). Thermodynamic analysis based on the simulated complex structure confirmed that the hydrophobic patches of F19/V36 in Aβ oligomer are the main contributors to recognize BDP-1 in aqueous environments.

### Example 5: BDP-1 Structural-Activity Relationship Study

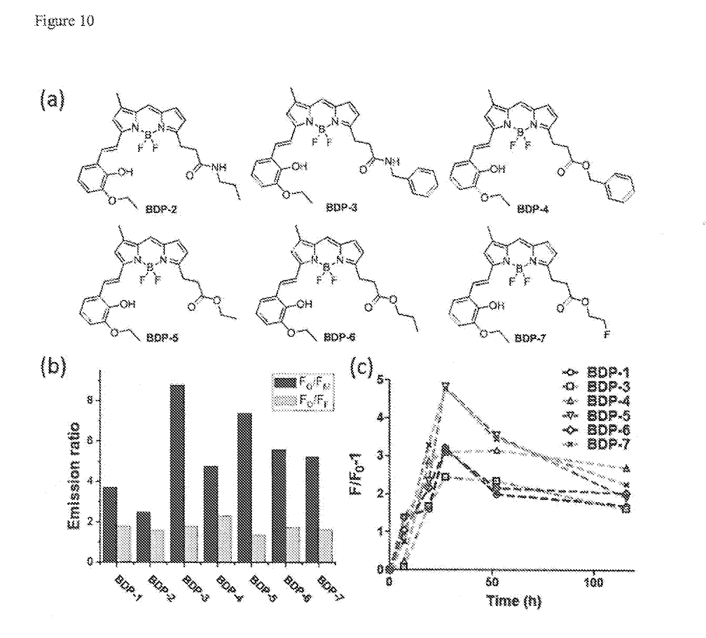

The building block was modified with similar structure on the compounds, to see whether similar changes on the structure can change the binding property with Aβ oligomers. For further structural modification, fluorine was introduced into the structure, which can be used for PET imaging in the future (FIG. 10a). The derivatives were tested against monomers, oligomers and fibrils of Aβ, and the ratio differences of the compound signal when reacted with oligomers were plotted against signal observed for either monomers or fibrils (FIG. 10b). Also their oligomer-sensing ability over the course of fibril formation was investigated. The derivatives displayed similar trends as the original compound, BDP-1, but with varying degrees of fluorescence fold-change (FIG. 10c).

### Example 6: BDP-1 and BDP-7 Labels Aβ Oligomers in AD Brain

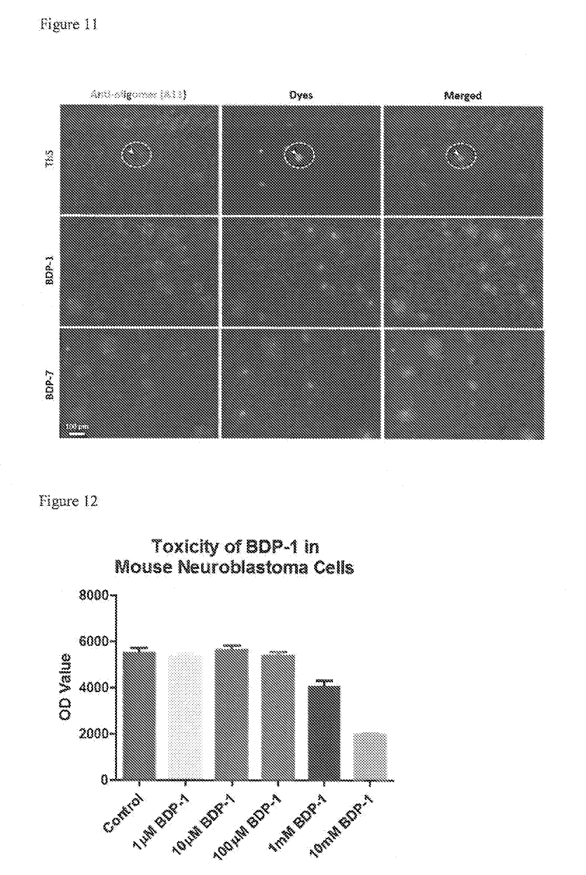

To investigate the oligomer detection ability of BDP-1 and its derivative in biological sample, a set of brain tissue imaging experiments were carried out. BDP-1, as the original Aβ oligomer sensor and BDP-7 which was modified for further PET study, were both chosen for further tissue testing. Staining with either BDP-1 or BDP-7 showed that both dyes have significant overlap with areas labeled by the A11 oligomer-specific antibody (FIG. 11). Also observed from the tissue staining was that both BDP-1 and BDP-7 displayed more intense staining in the core region which are not labeled by A11, synonymous with ThS stained areas. It was postulated that the tissue staining pattern was a reflection of this phenomena, where the probe labeled-soluble Aβ intermediates were associated with plaque cores, as well as with the periphery of plaques.

### Example 7: Toxicity of BDP-1 in Tissue Culture

The toxicity of BDP-1 was tested in N2a mouse neuroblastoma cells by CellTiter 96 AQueoue Non-Radioactive Cell Proliferation Assay (Promega, Madison, Wis.) as previously described (Chung E., et al. PLoS ONE, 2011, 6). Prior to the analysis, cells were seeded into 96-well plates in triplicate and allowed to attach overnight, before being treated with BDP-1 at concentrations of 1 μM to 10 mM for 72 hr. The MTS colorimetric solution [3-(4,5-dimethylthiazol-2-yl)-5-(3-carboxymethoxyphenyl)-2-(4-sulfophenyl)-2H-tetrazolium] was then added and bioreduced by cells into a soluble formazan product at 37° C. for 2-3 hours. The absorbance of the formazan at 490 nm was measured directly from 96-well plates in a Spectramax M2 plate reader using SoftMaxPro software Version 4.8. Cellular viability was determined as percent of control, with control being non-treated cells. Statistical significance of compound toxicity was analyzed by one way ANOVA followed by a post hoc Dunnett's multiple comparison test (GraphPad Prism, version 6.01; GraphPad Inc., San Diego, Calif., USA).

As shown in FIG. 12, BDP-1 was not toxic to the cells at concentrations even up to 100 μM, which is much higher than what would be used for in vivo applications, as typical concentrations that could be achieved in vivo in plasma would be ≦1 μM (Chung E., et al. PLoS ONE, 2011, 6).

### Example 8: In Vivo Detection of AD Oligomers by BDP-1 in AD Model Mice

The oligomer detection ability of BDP-1 in biological samples was further investigated. A stock solution of BDP-1 was made at 10 mM in DMSO. 18 month old APP/PS1 transgenic (Tg) AD model mice were given intraperitoneal (I.P.) injections with either 1.25 μl of BDP-1 diluted in 500 μl saline (n=2) or 500 μl of saline alone (n=2). This corresponded to a very low dose of BDP-1 of ˜7.15 μg/mouse or 0.28 mg/kg body weight. APP/PS1 Tg mice developed amyloid plaques from 4 months of age (Holcomb L., et al. Nature Medicine, 1998, 4, 97). Mice were anesthetized with an overdose of sodium pentobarbital and perfused with 0.1 M PBS (pH 7.4). Brains were removed 24 hr after the I.P. injection and fixed by immersion in periodate-lysine-paraformaldehyde for 24 hr, cryo-protected in 30% sucrose for 3 days and sectioned into 40 μm coronal sections using a cryostat. Brain sections from the BDP-1 injected mice and the control APP/PS1 mice were then stained for Aβ using fluorescent immunohistochemistry. Briefly, free floating sections were incubated with MOM blocking reagent (Vector) followed by an overnight incubation at 4° C. with anti-Aβ antibodies 4G8 and 6E10 diluted in MOM protein concentrate (Vector) as previously described (Scholtzova H., et al. Acta Neuropathol. Commun, 2014, 2, 101; Goni F., et al. Journal of Neuroinflammation, 2013, 10, 150). Sections were then incubated with a 488 conjugated secondary antibody (Jackson Immunoresearch) for 2 hr at room temperature, mounted onto slides and cover slipped. Staining was visualized using a LMD6500 fluorescent microscope (Leica); 6E10/4G8 staining was imaged in the green (488) channel and BDP-1 was imaged in the red (561) channel.

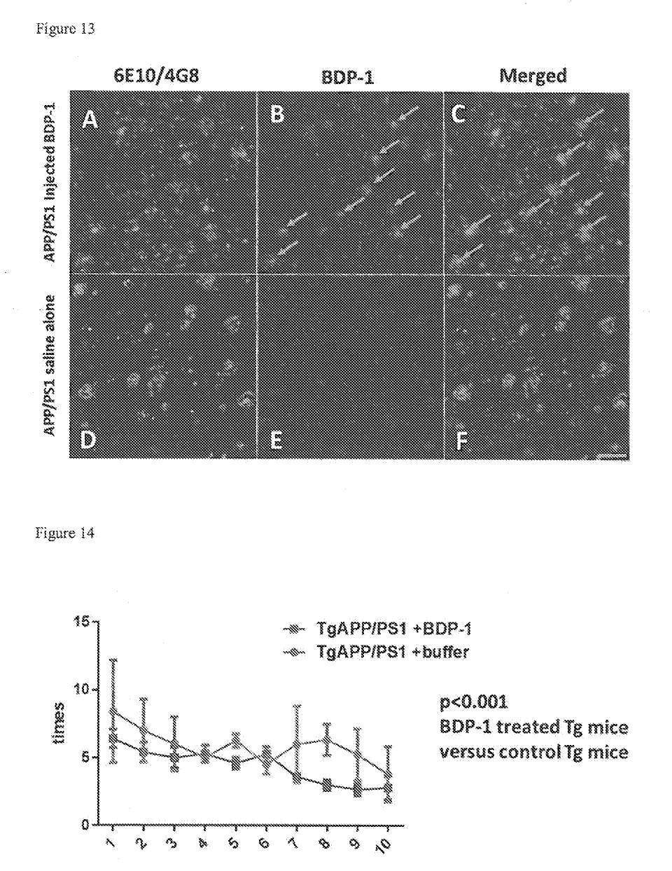

As shown in FIG. 13, I.P. injection of BDP-1 led to fluorescent labeling of plaques. BDP-1 labeling appeared strongest in the central core of the plaques, but there was also labeling of less compacted amyloid present around the periphery of plaques (see FIG. 13B). The labeling of BDP-1 co-localized with the labeling using anti-Aβ antibodies 4G8/6E10 (see FIG. 13C). Fluorescent plaque staining was not present on the APP/PS1 mouse injected with saline alone (see FIG. 13E). There also appeared to be some punctate, possibly intraneuronal, staining with BDP-1 surrounding plaques, which had brighter intensity than the endogenous autofluorescence present in the control APP/PS1 brains. It should be noted that a very low dose of BDP-1 (0.28 mg/kg) was used in this imaging experiment, whereas other amyloid imaging agents published were used at doses of ˜30 mg/kg (Li Q., et al. Chembiochem, 2007, 8, 1679), suggesting the high degree of in vivo avidity and specificity of BDP-1.

Taken together, BDP-1 successfully penetrated the BBB to show Aβ oligomers detection capabilities in the brains of AD transgenic mice model without toxicity even when used at very low concentrations.

### Example 9: Testing of BDP-1 as a Therapeutic Agent in AD Model Mice

It was further tested whether BDP-1 can be given chronically by I.P. injection in AD model mice and whether this will be associated with cognitive benefits.

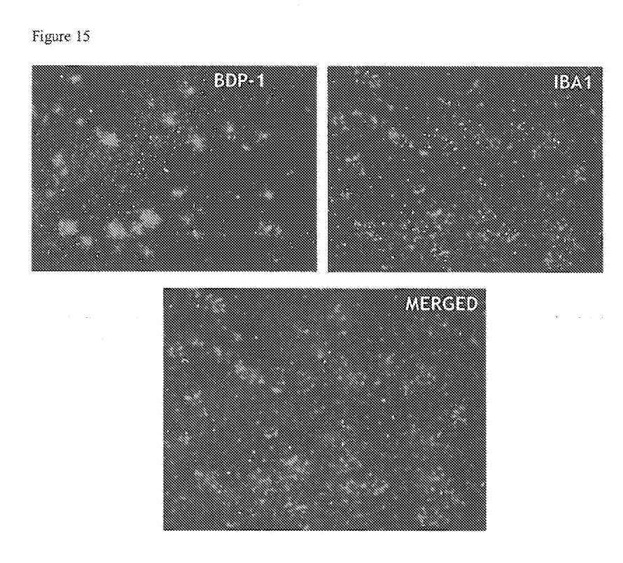

To this end, two groups of 11 APP K670N/M671L/PS1 M146L (APP/PS1) Tg mice (Holcomb L., et al. Nature Medicine, 1998, 4, 97) were used from the age of 4 months with one group being give BDP-1 at a dose of 0.7 μg/mouse by I.P. injection twice per week over a period of 2 months. This dose of BDP-1 corresponded to 0.03 mg/kg body weight, and was much lower (˜1/1,000 to 1/100) than the doses of other peptides/small compounds administered to AD model mice (Sadowski M., et al. PNAS, 2006, 103, 18787). The other group was given I.P. injections of vehicle alone. The mouse breeding and genotyping were as previously described (Goni F, et al. Journal of Neuroinflammation, 2013, 10, 150; Sadowski M, et al. PNAS, 2006, 103, 18787; Goni F, et al. PLoS. ONE, 2010, 5). Amyloid deposition in this mouse AD Tg model starts at about the age of 3 months (McGowan E, et al. Neurobiology of Disease, 1999, 6, 231). After one month of BDP-1 treatment one of the mice in the treatment group was sacrificed and the brain was processed as above to assess if BDP-1 was crossing the BBB and binding to oligomers. The brain sections were also labeled immunohistochemically with antibodies to IBA1, a marker of microglia, to examine the colocalization of BDP-1 and microglia (FIG. 15). At the age of 6 months the two groups of Tg mice were subjected to sensorimotor and behavioral testing using radial arm maze.

Sensorimotor and cognitive testing were done as previously described (Scholtzova H, et al. Acta Neuropathol. Commun, 2014, 2, 10; Sadowski M, et al. PNAS, 2006, 103, 18787; Scholtzova H, et al. J. Neurosci. Res, 2008, 86 2784; Asuni A, et al. Eur. J Neurosci, 2006, 24, 2530). Prior to testing, the mice were adapted to the room with lights on for 15 min. The main objective of performing these sensorimotor tasks was to verify that any treatment related effects observed in the cognitive tasks could not be explained by differences in sensorimotor abilities.

Locomotor Activity:

A Hamilton-Kinder Smart-frame Photobeam System was used to make a computerized recording of animal activity over a designated period of time. Exploratory locomotor activity was recorded in a circular open field activity measuring chamber (70×70 cm). A video camera mounted above the chamber automatically recorded horizontal movements in the open field in each dimension (i.e., x, y, and two z planes). Total distance was measured in centimeters (cm) traveled and was defined as sequential movement interruptions of the animal measured relative to the background. The duration of the behavior was timed for 15 min. Results were reported based on distance traveled (cm), mean resting time, and maximum velocity of the animal.

Traverse Beam:

This task tested balance and general motor coordination and function integration. Mice were assessed by measuring their ability to traverse a graded narrow wooden beam to reach a goal box specifically examining hind limb function. The mice were placed on a 1.1 cm wide beam 50.8 cm long suspended 30 cm above a padded surface by two identical columns. Attached at each end of the beam was a shaded goal box. Mice were placed on the beam in a perpendicular orientation to habituate, and were then monitored for a maximum of 60 sec. The number of foot slips each mouse had before falling or reaching the goal box was recorded for each of three successive trials. The average foot slips for all four trials was calculated and recorded. Errors were defined as foot slips and recorded both numerically and using Feeney scores. To prevent injury from falling, a soft foam cushion was always kept underneath the beam. Animals that fell off were placed back in their position prior to the fall.

Rotarod:

The animal was placed onto the rod (diameter 3.6 cm) apparatus to assess differences in motor coordination and balance by measuring fore- and hind limb motor coordination and balance (Rotarod 7650 accelerating model; Ugo Basile, Biological Research Apparatus, Varese, Italy). This procedure was designed to assess motor behavior without a practice confound. The animals were habituated to the apparatus by receiving training sessions of two trials, sufficient to reach a baseline level of performance. Then the mice were tested a further 3 times, with increasing speed. During habituation, the rotor rod was set at 1.0 rpm, which was gradually raised every 30 sec, and was also wiped clean with 30% ethanol solution after each session. A soft foam cushion was placed beneath the apparatus to prevent potential injury from falling. Each animal was tested for three sessions, with each session separated by 15 min, and measures were taken for latency to fall or invert (by clinging) from the top of the rotating barrel.

Radial Arm Maze:

Spatial learning was evaluated using an eight-arm radial maze with a water well at the end of each arm. Clear Plexiglas guillotine doors, operated by a remote pulley system, controlled access to the arms from a central area from which the animals entered and exited the apparatus. After 4 days of adaptation to the maze, water-restricted mice (2 h daily access to water) were given one training session per day for ten consecutive days. This relatively long adaptation period was used as it was found that these Tg AD mice tend to be very anxious and will not run the maze well without adaptation (Sadowski M, et al. PNAS, 2006, 103, 18787; Asuni A, et al. Eur. J Neurosci, 2006, 24, 2530). Prior to each day's testing, the mice were adapted to the room with lights on for 15 min. For each session, all arms were baited with saccharine flavored water, and animals were permitted to enter all arms until the eight rewards had been consumed. The number of errors (entries to previously visited arms) and time to complete each session were recorded.

Results: No differences in locomotor activity were noted in the two groups of Tg mice (data not shown). However, the BDP-1 treated mice showed a clear cognitive benefit with fewer errors on the radial arm maze compared to the vehicle-treated Tg mice, as shown in FIG. 14 (p<0.001, by Bonferroni's multiple comparisons test).

### Example 10: Testing of BDP-1 for Anti-Prion Infection Activity

It was known that toxic oligomeric species of many of the proteins involved in different neurodegenerative conditions have structural similarities (Wisniewski T, et al. Neuron, 2015, 85, 1162; Glabe C G. J Biol. Chem, 2008, 283, 29639). It was also known that this structural similarity allows the potential use of a therapeutic approach targeting one type of oligomer to also target oligomers with a completely different protein sequence (Goni F, et al. Journal of Neuroinflammation, 2013, 10, 150; Wisniewski T, et al. Neuron, 2015, 85, 1162). Hence it was further tested whether BDP-1 could be used to inhibit PrP^{Sc} infection in a tissue culture model, a well-established model system of prion infection (Chung E, et al. PLoS ONE, 2011, 6; Pankiewicz J, et al. Eur. J Neurosci, 2006, 24, 2635).

To this end, N2a mouse neuroblastoma cells were maintained in minimal essential medium (MEM) supplemented with heat-inactivated 10% fetal bovine serum, penicillin (100 U/ml) and streptomycin (100 μg/ml) at 37° C. in 5% CO₂. Brains of terminally ill CD-1 mice infected with mouse-adapted 22L prion strain were homogenized (10% w/v) in cold phosphate-buffered saline and 5% sucrose under sterile conditions, as previously described (Chung E, et al. PLoS ONE, 2011, 6; Pankiewicz J, et al. Eur. J Neurosci, 2006, 24, 2635; Sadowski M J, et al. Neurobiol Dis, 2009, 34, 267).

For infection of N2a cells, the homogenate was further diluted to 2% in Opti-MEM and added to confluent 12.5 cm² flasks (Falcon). After 4-5 hours, an equal volume of regular MEM was added and cells were incubated in the presence of infectious brain homogenate overnight. The cells were then washed twice with PBS and fresh MEM was added. Cells were allowed to grow until confluence and then were split into 1:4 dilutions and transferred to 25-cm² flasks until the fourth passage when traces of 22L brain homogenate can no longer be detected. N2a/22L cells (from the fifth passage after infection and above) were plated in six-well plates. BDP-1 was applied at concentrations ranging from 0.5 to 10 μM for 72 hr. A fresh treatment was applied daily until lysis. The level of PK-resistant PrP^{Sc} was measured by Western blot. Each experiment included a positive control (non-treated N2a/22L cells) and a negative control (non-infected N2a cells). Anti-PrP 6D11 was applied at a concentration of 1 μg/ml for 72 h as a treatment positive control (Pankiewicz J, et al. Eur. J Neurosci, 2006, 24, 2635). For detection and quantification of PrP^{Sc} in N2a/22L cells were harvested using ice-cold lysis buffer [NaCl, 150 mM; triton X-100, 0.5%; sodium deoxycholate, 0.5%; and Tris-HCl, 50 mM, pH 7.5; with a protease inhibitor cocktail (Roche, Indianapolis, Ind., USA)], as previously described (Chung E, et al. PLoS ONE, 2011, 6; Pankiewicz J, et al. Eur. J Neurosci, 2006, 24, 2635; Sadowski M J, et al. Neurobiol Dis, 2009, 34, 267). The lysates were centrifuged for 3 min at 10,000 g to remove cell debris and the total protein concentration was measured in the supernatant using the bicinchoninic acid assay (BCA; Pierce, Rockford, Ill., USA). Aliquots containing 200 μg of total protein were titrated by adding buffer to achieve a final protein concentration of 1 μg/μl. Samples were digested with proteinase K (PK; Roche) for 30 min at 37° C. The enzyme-to-protein weight ratio was 1:50. PK activity was quenched by adding phenylmethanesulphonyl fluoride to achieve a final concentration of 3 mM. Samples were then centrifuged at 20,000 g for 45 min at 4° C. Pellets were resuspended in PBS and tricine sample buffer (Bio-rad, Hercules, Calif., USA) with β-mercaptoethanol (BME), boiled at 95° C. for 5 min and then subjected to electrophoresis on 12.5% SDS-polyacrylamide Tris-tricine gels. Following overnight electrophoresis the proteins were transferred onto nitrocellulose membranes (Amersham Biosciences, Piscataway, N.J., USA) for 1 hr at 400 mÅ using CAPS buffer (3-cyclohexylamino-1-propanesulphonic acid) containing 10% methanol. The membranes were blocked with 5% Carnation nonfat milk in TBST (Tris, 10 mM; NaCl, 150 mM; Tween 20, 0.1%, pH 7.5) for 1 hr at room temperature and then incubated with anti-PrP Mab 6D11 diluted to 1:3000 (Spinner D S, et al. J Leukoc. Biol, 2007, 14, 36). Following extensive washing in TBST the membranes were incubated with a horseradish-peroxidase conjugated goat anti-mouse antibody (Thermo Scientific, Rockford, Ill., USA) and then developed using an enhanced chemiluminescent substrate (ECL Western Blotting Substrate; Pierce). Membranes were applied to autoradiography film (Super RX Fuji Medical XRay Film; Fujifilm, Tokyo, Japan). Developed films were converted into 8-bit grayscale digital files using an Epson Perfection 1200U scanner (Epson America, Long Beach, Calif., USA) and Adobe Photoshop software (Adobe Systems, San Jose, Calif., USA) and saved in JPEG format with a resolution of 600 dpi. Quantification of PrP^{Sc} was performed by densitometric analysis using NIH Image J software. Areas under the curves for the three PrP bands representing non-, mono-, and diglycosylated isoforms of the protein were summarized from each sample to calculate total PrP^{Sc} level.

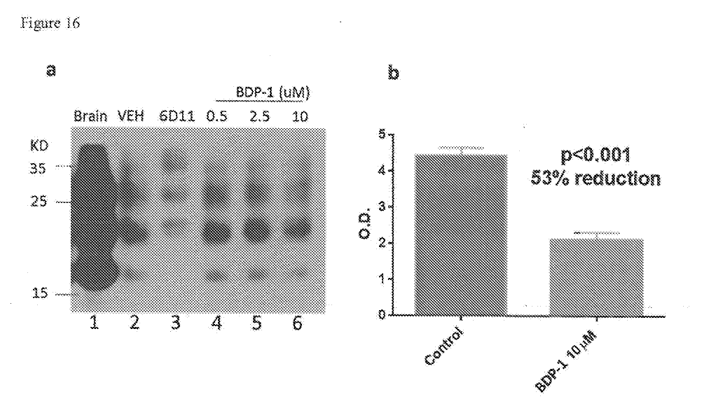

As shown in FIG. 16, treatment of BDP-1 at 10 μM concentration (comparing lane 6 to lane 2) produced a ˜50% reduction in PrP^{Sc} infection of the cells. FIG. 16B summarizes 3 independent replicates of the experiment comparing the densities of the bands of vehicle treated N2a/22L cells (lane 2 in FIG. 16A) to N2a/22L cells treated with 10 μM BDP-1 (p<0.001, by two-tailed student's t-test). Therefore, BDP-1 successfully inhibited prion infection and has the potential to be used as a therapeutic agent in prion diseases.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. The word “comprise” or variations such as “comprises” or “comprising” will accordingly be understood to imply the inclusion of a stated integer or groups of integers but not the exclusion of any other integer or group of integers. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The content of all documents and patent documents cited herein is incorporated by reference in their entirety.

1. A compound of Formula (I) or selected from the group consisting of BDP-1, BDP-4, BDP-5, BDP-6, and BDP-7, 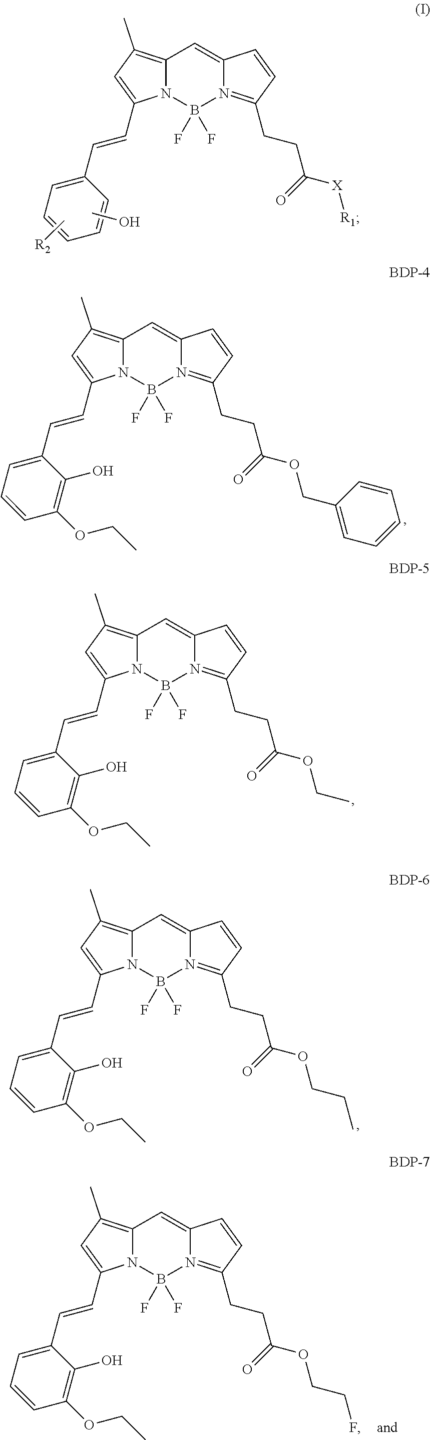 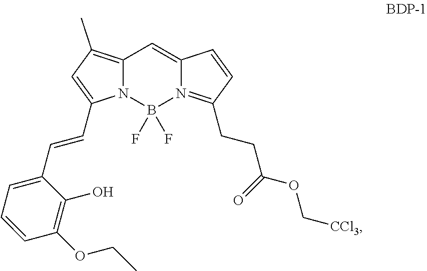
wherein:
X is a direct bond or is selected from the group consisting of —NR—, and —S—;
R is selected from the group consisting of H and a substituted or unsubstituted alkyl; and
R₁ and R₂ are each independently selected from the group consisting of H, halogen, a substituted or unsubstituted alkyl, a substituted or unsubstituted alkoxy, a substituted or unsubstituted alkenyl, a substituted or unsubstituted alkynyl, a substituted or unsubstituted heterocycle, a substituted or unsubstituted aryl, a substituted or unsubstituted alkyl-aryl, a substituted or unsubstituted alkyl-heterocycle, a substituted or unsubstituted heteroaryl.

2. The compound of claim 1, wherein X is —NH—.

3. The compound of claim 1, wherein R₁ is a substituted or unsubstituted C₁-C₁₀ alkyl.

4. The compound of claim 3, wherein R₁ is ethyl or propyl.

5. The compound of claim 3, wherein R₁ is —CH₂CH₂F.

6. The compound of claim 3, wherein R₁ is —CH₂CCl₃.

7. The compound of claim 1, wherein R₁ is a substituted or unsubstituted C₇-C₁₀ alkyl-aryl.

8. The compound of claim 7, wherein R₁ is benzyl.

9. The compound of claim 1, wherein R₂ is at the 3-position (meta) and OH is at the 2-position (ortho) of the phenyl ring.

10. The compound of claim 1, wherein R₂ is a substituted or unsubstituted C₁-C₁₀ alkoxy.

11. The compound of claim 10, wherein R₂ is ethoxy.

12. The compound of claim 1, wherein the compound of Formula (I) is selected from the group consisting of BDP-2 and BDP-3,

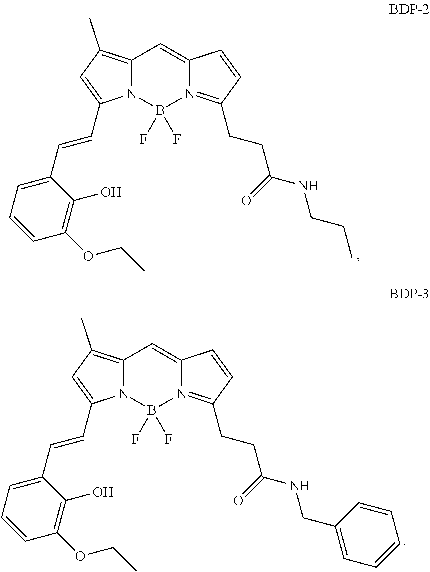

13. A method for selectively detecting oligomeric proteins or peptides associated with a conformational disorder in a sample, the method comprising:
(a) contacting the sample with a compound of Formula (I), or a conjugate comprising said compound and a detectable marker, wherein the compound binds selectively to the oligomers, under conditions that allow binding of the compound to the oligomers, 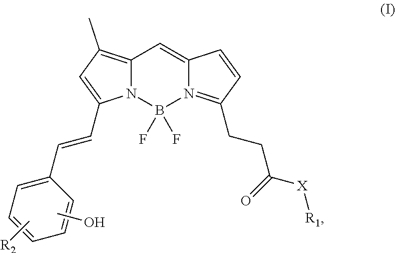
wherein:
X is a direct bond or is selected from the group consisting of —NR—, —O—, and —S—;
R is selected from the group consisting of H and a substituted or unsubstituted alkyl; and
R₁ and R₂ are each independently selected from the group consisting of H, halogen, a substituted or unsubstituted alkyl, a substituted or unsubstituted alkoxy, a substituted or unsubstituted alkenyl, a substituted or unsubstituted alkynyl, a substituted or unsubstituted heterocycle, a substituted or unsubstituted aryl, a substituted or unsubstituted alkyl-aryl, a substituted or unsubstituted alkyl-heterocycle, a substituted or unsubstituted heteroaryl; and
(b) detecting the protein oligomers by measuring the signal of the complexes formed between the oligomers and said compound or conjugate.

14. The method of claim 13, wherein the oligomeric proteins or peptides are AP oligomers or PrP^{Sc} oligomers.

15. A method for diagnosing a conformational disorder associated with protein or peptide oligomers in a subject, comprising administering to said subject an effective amount of a compound of Formula (I), or a conjugate comprising said compound and a detectable marker, and measuring the signal of the complexes formed between said oligomers and said compound or conjugate, 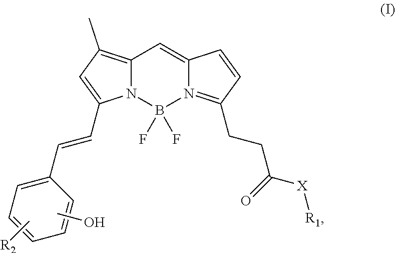
wherein:
X is a direct bond or is selected from the group consisting of —NR—, —O—, and —S—;
R is selected from the group consisting of H and a substituted or unsubstituted alkyl; and
R₁ and R₂ are each independently selected from the group consisting of H, halogen, a substituted or unsubstituted alkyl, a substituted or unsubstituted alkoxy, a substituted or unsubstituted alkenyl, a substituted or unsubstituted alkynyl, a substituted or unsubstituted heterocycle, a substituted or unsubstituted aryl, a substituted or unsubstituted alkyl-aryl, a substituted or unsubstituted alkyl-heterocycle, a substituted or unsubstituted heteroaryl.

16. A method for treating or preventing a conformational disorder associated with protein or peptide oligomers in a subject, comprising administering to said subject an effective amount of a compound of Formula (I), or a pharmaceutical composition comprising said compound or a conjugate comprising said compound and a detectable marker and a pharmaceutically acceptable carrier, 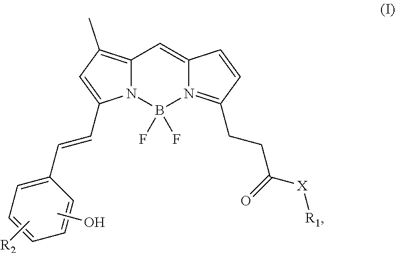
wherein:
X is a direct bond or is selected from the group consisting of —NR—, —O—, and —S—;
R is selected from the group consisting of H and a substituted or unsubstituted alkyl; and
R₁ and R₂ are each independently selected from the group consisting of H, halogen, a substituted or unsubstituted alkyl, a substituted or unsubstituted alkoxy, a substituted or unsubstituted alkenyl, a substituted or unsubstituted alkynyl, a substituted or unsubstituted heterocycle, a substituted or unsubstituted aryl, a substituted or unsubstituted alkyl-aryl, a substituted or unsubstituted alkyl-heterocycle, a substituted or unsubstituted heteroaryl.

17. The method of claim 15, wherein the disorder is a neurodegenerative disorder.

18. The method of claim 17, wherein the neurodegenerative disorder is Alzheimer's Disease (AD) or a prion disease.

19. The method of claim 15, wherein the subject is a mammal.

20. The method of claim 16, wherein the disorder is a neurodegenerative disorder.

21. The method of claim 16, wherein the subject is a mammal.


SEQUENCE LISTING (12 sequences)

SEQ ID NO: 1 - protein, 40 aa, Homo sapiens
DAEFRHDSGYEVHHQKLVFFAEDVGSNKGAIIGLMVGGVV

SEQ ID NO: 2 - protein, 42 aa, Homo sapiens [sequence omitted]

SEQ ID NO: 3 - protein, 43 aa, Homo sapiens
DAEFRHDSGYEVHHQKLVFFAEDVGSNKGAIIGLMVGGVVIAT

SEQ ID NO: 4 - protein, 41 aa, Homo sapiens
DAEFRHDSGYEVHHQKLVFFAEDVGSNKGAIIGLMVGGVVI

SEQ ID NO: 5 - protein, 38 aa, Homo sapiens
EFRHDSGYEVHHQKLVFFAEDVGSNKGAIIGLMVGGVV

SEQ ID NO: 6 - protein, 40 aa, Homo sapiens
EFRHDSGYEVHHQKLVFFAEDVGSNKGAIIGLMVGGVVIA

SEQ ID NO: 7 - protein, 39 aa, Homo sapiens
FRHDSGYEVHHQKLVFFAEDVGSNKGAIIGLMVGGVVIA

SEQ ID NO: 8 - protein, 34 aa, Homo sapiens
GYEVHHQKLVFFAEDVGSNKGAIIGLMVGGVVIA

SEQ ID NO: 9 - protein, 32 aa, Homo sapiens
EVHHQKLVFFAEDVGSNKGAIIGLMVGGVVIA

SEQ ID NO: 10 - protein, 20 aa, Homo sapiens
LVFFAEDVGSNKGAIIGLMV

SEQ ID NO: 11 - protein, 209 aa, Homo sapiens
KKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSS

SEQ ID NO: 12 - protein, 253 aa, Homo sapiens
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG